(19) United States
(12) Patent Application Publication
(10) Pub. No.: US 2025/0019447 A1
(43) Pub. Date: Jan. 16, 2025

(54) COMBINATION THERAPY FOR TREATMENT OF CANCER

(71) Applicant: AVEO Pharmaceuticals, Inc., Boston, MA (US)

(72) Inventor: Julie E. Bauman, Tuscon, AZ (US)

(21) Appl. No.: 18/667,500

(22) Filed: May 17, 2024

Related U.S. Application Data
(63) Continuation of application No. PCT/US2021/059975, filed on Nov. 18, 2021.

(51) Int. Cl. C07K 16/28; A61K 39/00; A61P 35/00; C07K 16/22

(52) U.S. Cl. CPC C07K 16/2863; A61P 35/00; C07K 16/22; A61K 2039/507; A61K 2039/54; A61K 2039/545; C07K 2317/21; C07K 2317/24

(57) ABSTRACT

The present invention provides improved methods of treating subjects with cancers, such as head and neck squamous cell carcinoma (HNSCC). In one aspect, the invention provides a method of treating cancer in a subject in need thereof. In some embodiments, the method comprises administering to the subject a therapeutically effective amount of an HGF inhibitor and an EGFR inhibitor. In some embodiments, recurrent or metastatic HNSCC is treated with a combination of ficlatuzumab and cetuximab.

### CROSS-REFERENCE TO RELATED APPLICATIONS

This Application is a Continuation of PCT/US2021/059975, filed on Nov. 18, 2021, and incorporated herein by reference in its entirety.

### SUBMISSION OF SEQUENCE LISTING XML

The content of the following submission on Sequence Listing XML is incorporated herein by reference in its entirety: a computer readable form (CRF) of the Sequence Listing (file name: 406767-AVO-044US-210182-sequence listing-2024_07_17.XML, date created Jul. 17, 2024, size: 1,785,486,784 bytes).

### FIELD OF THE INVENTION

The field of the invention is medicine, oncology, tyrosine kinase inhibitors, EGFR receptor inhibitors, and pharmaceuticals.

### BACKGROUND

Head and neck squamous cell carcinoma (HNSCC) is a morbid and lethal cancer caused by habitual exposure to tobacco and other carcinogens or by the human papillomavirus (HPV). Head and neck squamous cell carcinoma (HNSCC) is the most common cancer arising in the upper aerodigestive tract. HNSCC is the sixth leading incident cancer worldwide with 600,000 cases anticipated in 2012 (Kamangar, F. et al. Journal of Clinical Oncology (2006) 24:2137-2150). Despite advances in multimodality therapy, 5-year overall survival (OS) is 40-50%, and has increased only incrementally in the past two decades (Jemal, A. et al. CA Cancer J Clin. (2010) 60:277-300). Patients with recurrent or metastatic (R/M) HNSCC have particularly poor prognosis, with median overall survival of 6-10 months. Options for palliative management are limited. For nearly three decades, the cornerstone of first line chemotherapy for R/M HNSCC has been a platinum-based antineoplastic drug (platin), such as cisplatin (Hong, W. K. et al. Cancer (1983) 52:206-210), frequently combined with fluorouracil or a taxane derivative due to increased response rate (RR) albeit no conclusive evidence of superior survival compared to cisplatin monotherapy (Forastiere, A. A. et al. J Clin Oncol. (1992) 10:1245-1251).

Ubiquitous expression of EGFR compelled the development of EGFR inhibitors for HNSCC treatment (Rubin Grandis, J. et al. J Natl Cancer Inst. (1998) 90:824-832; Chung, C. H. et al. J Clin Oncol. (2006) 24:4170-4176). The EGFR-directed monoclonal antibody, cetuximab, is the only targeted therapy to date FDA-approved for the treatment of HNSCC, and improves survival when added to front line platinum (Vermorken, J. B. et al. N Engl J Med. (2008) 359:1116-1127). Despite aberrant EGFR signaling in the majority of HNSCC cases, the modest clinical activity of cetuximab has been disappointing; either primary or acquired resistance is an overwhelmingly common occurence. Currently, there is no predictive molecular marker for resistance or sensitivity to anti-EGFR therapy in HNSCC, including EGFR gene copy number (Licitra, L. et al. Ann Oncol. (2011) 22:1078-1087).

While immunotherapeutic antibodies inhibiting programmed death receptor 1 (PD-1) recently gained FDA approval in patients with platinum-resistant HNSCC, the overall survival (OS) benefit appears to be limited to approximately 20% (Seiwert, T. Y. et al. Lancet Oncol. (2016) 17:956-965; Ferris, R. L. et al. J Clin Oncol. (2016) 34). Currently, there is no standard therapy for patients after failure of platinum, cetuximab, and anti-PD1 therapy; all such patients will succumb with a median survival of less than 6 months.

Accordingly, there exists a need for further therapeutic options for patients with HNSCC, including recurrent or metastatic HNSCC.

### SUMMARY OF THE INVENTION

The present invention provides improved methods of treating subjects with cancers, such as head and neck squamous cell carcinoma (HNSCC).

In one aspect, the invention provides a method of treating recurrent or metastatic head and neck squamous cell carcinoma (HNSCC) in a subject. The method involves identifying a subject having recurrent or metastatic HNSCC that is human papillomavirus (HPV) negative, and administering to the subject an effective amount of cetuximab with an effective amount of ficlatuzumab, thereby to treat the HNSCC that is HPV negative. In some embodiments, if the subject has recurrent or metastatic HNSCC that is HPV positive, the subject is not treated. In some embodiments, the HNSCC has not been previously treated with cetuximab, i.e., the HNSCC is cetuximab naïve.

In some embodiments, the HPV status of the HNSCC is determined by p16 immunohistochemistry. For example, the HNSCC is classified as HPV negative if the HNSCC is p16 negative. For example, the HNSCC is classified as HPV negative if the HNSCC is primary oral, laryngeal, or hypopharyngeal HNSCC. For example, the HNSCC is classified as HPV positive if the HNSCC is p16 positive. For example, the HNSCC is classified as HPV positive if the HNSCC is primary site oropharyngeal HNSCC and the HNSCC is p16 positive. In some embodiments, the HPV status is determined by tumoral DNA analysis. For example, the HNSCC is HPV negative if HPV DNA or RNA is not detected in the HNSCC. For example, the HNSCC is HPV positive if HPV DNA or RNA is detected in the HNSCC.

In some embodiments, the HNSCC in the subject has been previously treated with immunotherapy such as a PD-1 or PD-L1 checkpoint inhibitor. In some embodiments, the HNSCC is PD-1 or PD-L1 immunotherapy-resistant. In some embodiments, the immunotherapy is selected from pembrolizumab, nivolumab, cemiplumab, atezolizumab, avelumab, durvalumab, ipilimumab, tremelimumab or tisotumab. In some embodiments, the immunotherapy is pembrolizumab. In some embodiments, the immunotherapy is nivolumab.

In some embodiments, the HNSCC was previously treated with platinum chemotherapy. In some embodiments, the HNSCC is platinum resistant or the subject is ineligible for platinum chemotherapy. In some embodiments, the platinum chemotherapy is carboplatin, oxaliplatin, cisplatin, nedaplatin, triplatin tetranitrate, phenanthriplatin, picoplatin, or satraplatin In some embodiments, the platinum therapy is carboplatin or cisplatin.

In some embodiments, the HNSCC in the subject was previously treated with 5-fluorouracil or another antimetabolite chemotherapy. In some embodiments, the HNSCC was previously treated with pembrolizumab, with or without platinum, and with or without 5-fluorouracil as a first line therapy.

In some embodiments, the HNSCC is from a primary site in the oral cavity, pharynx, or larynx. In some embodiments, the HNSCC is hypopharyngeal cancer, laryngeal cancer, lip or oral cavity cancer, nasopharyngeal cancer, oropharyngeal cancer, paranasal sinus or nasal cavity cancer, or salivary gland cancer.

In some embodiments, the dose of ficlatuzumab is 10 mg/kg to 100 mg/kg. In some embodiments, the dose of ficlatuzumab is 10 mg/kg to 50 mg/kg. In some embodiments, the dose of ficlatuzumab is 20 mg/kg. In some embodiments, the dose of ficlatuzumab is 15 mg/kg. In some embodiments, the dose of ficlatuzumab is 10 mg/kg. In some embodiments, the dose of ficlatuzumab is administered every two weeks by intravenous administration. In some embodiments, the dose of ficlatuzumab is administered every two weeks +/−3 days by intravenous administration.

In some embodiments, the dose of cetuximab is 250 to 700 mg/m². In some embodiments, the dose of cetuximab is 300 to 500 mg/m². In some embodiments, the dose of cetuximab is 500 mg/m². In some embodiments, the dose of cetuximab is 400 mg/m². In some embodiments, the dose of cetuximab is 300 mg/m². In some embodiments, the dose of cetuximab is administered every two weeks by intravenous administration. In some embodiments, the dose of cetuximab is administered every two weeks +/−3 days by intravenous administration. In some embodiments, the dose of cetuximab and the dose of ficlatuzumab are administered on the same day, either simultaneously or sequentially by intravenous administration.

In some embodiments, the dose of cetuximab is 500 mg/m² and the dose of ficlatuzumab is 20 mg/kg, each administered on the same day, either simultaneously or sequentially by intravenous infusion every two weeks.

In some embodiments, the subject is administered cetuximab and ficlatuzumab until progression of the HNSCC, development or a new metastasis, or the patient experience unacceptable toxicity.

In another aspect, the invention provides a method of identifying a subject with head and neck squamous cell carcinoma (HNSCC) that is suitable for a combination therapy with ficlatuzumab and cetuximab. In some embodiments, the method of identifying comprises assessing whether the HNSCC is human papillomavirus (HPV)-negative or HPV-positive. In some embodiments, the subject is identified as suitable for the combination therapy if the HNSCC is human papillomavirus (HPV)-negative.

In some embodiments, the subject is identified as suitable for the combination therapy if the HNSCC is platinum-resistant or the subject is ineligible for platinum chemotherapy.

In some embodiments, the subject is identified as suitable for the combination therapy if the HNSCC is not responsive to immunotherapy with a PD-1 or PD-L1 inhibitor.

In some embodiments, the subject is identified as suitable for the combination therapy if the subject is ineligible for immunotherapy, such as with a PD-1 or PD-L1 inhibitor

In some embodiments, the subject is identified as suitable for the combination therapy if the HNSCC is platinum-resistant and not responsive to immunotherapy with a PD-1 or PD-L1 inhibitor. In some embodiments, the subject is identified as suitable for the combination therapy if the immunotherapy is pembrolizumab, nivolumab, cemiplumab, atezolizumab, avelumab, durvalumab, ipilimumab, tremelimumab or tisotumab. In some embodiments, the subject is identified as suitable for the combination therapy if the immunotherapy is pembrolizumab or nivolumab. In some embodiments, the subject is identified as suitable for the combination therapy if the platinum is carboplatin or cisplatin.

In some embodiments, the method of identifying further comprises administering the combination therapy to the subject if the subject is identified as suitable for combination therapy.

In some embodiments, HPV status of the HNSCC is determined by p16 immunohistochemistry. In some embodiments, the HNSCC is HPV negative when the HNSCC is classified as p16 negative. In some embodiments, the HNSCC is HPV positive when the HNSCC is classified as p16 positive. In some embodiments, an HPV status of the HNSCC is determined by tumoral DNA analysis. For example, the HNSCC is HPV negative if HPV DNA is not detected in the HNSCC. For example, the HNSCC is HPV positive if HPV DNA is detected in the HNSCC.

In some embodiments, the HNSCC is from a primary site in the oral cavity, pharynx, or larynx. In some embodiments, the HNSCC is hypopharyngeal cancer, laryngeal cancer, lip or oral cavity cancer, nasopharyngeal cancer, oropharyngeal cancer, paranasal sinus or nasal cavity cancer, or salivary gland cancer.

In some embodiments, the combination therapy is a first line therapy. In some embodiments, the combination therapy is a second line therapy. In some embodiments, the combination therapy is a third line or later therapy.

In some embodiments, the combination therapy comprises administering a dose of ficlatuzumab that is 10 mg/kg to 100 mg/kg. In some embodiments, the combination therapy comprises administering a dose of ficlatuzumab that is 10 mg/kg to 50 mg/kg. In some embodiments, the combination therapy comprises administering a dose of ficlatuzumab that is 20 mg/kg. In some embodiments, the combination therapy comprises administering a dose of ficlatuzumab that is 15 mg/kg. In some embodiments, the combination therapy comprises administering a dose of ficlatuzumab that is 10 mg/kg.

In some embodiments, the combination therapy comprises administering a dose of cetuximab that is 250 to 700 mg/m². In some embodiments, the combination therapy comprises administering a dose of cetuximab that is 300 to 500 mg/m². In some embodiments, the combination therapy comprises administering a dose of cetuximab that is 500 mg/m². In some embodiments, the combination therapy comprises administering a dose of cetuximab that is 400 mg/m². In some embodiments, the combination therapy comprises administering a dose of cetuximab that is 300 mg/m².

In some embodiments, the combination therapy comprises administering a dose of ficlatuzumab and cetuximab every two weeks by intravenous administration. In some embodiments, the combination therapy comprises administering a dose of ficlatuzumab and cetuximab every two weeks +/−3 days by intravenous administration.

In some embodiments, the combination therapy comprises administering a dose of cetuximab and a dose of ficlatuzumab on the same day, for example, either simultaneously or sequentially

In one aspect, the invention provides a method of treating cancer in a subject in need thereof. The method comprises administering to the subject a therapeutically effective amount of an HGF inhibitor and an EGFR inhibitor.

In certain embodiments, the HGF inhibitor is an anti-HGF antibody or antigen binding fragment thereof. In certain embodiments, the anti-HGF antibody or antigen binding fragment thereof comprises: (i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and/or (ii) an immunoglobulin light chain variable region comprising a CD_{RL1} comprising the amino acid sequence of SEQ ID NO: 4, a CDR_{L2} comprising the amino acid sequence of SEQ ID NO: 5, and a CDR_{L,3} comprising the amino acid sequence of SEQ ID NO: 6. In certain embodiments, the anti-HGF antibody or antigen binding fragment thereof comprises: (i) an immunoglobulin heavy chain variable region comprising the amino acid sequence of SEQ ID NO: 7, or an amino acid sequence having at least 80%, 85%, 90%, 95%, 96%, 97%, 98%, or 99% identity to the amino acid sequence of SEQ ID NO: 7; and (ii) an immunoglobulin light chain variable region comprising the amino acid sequence of SEQ ID NO: 8, or an amino acid sequence having at least 80%, 85%, 90%, 95%, 96%, 97%, 98%, or 99% identity to the amino acid sequence of SEQ ID NO: 8. In certain embodiments, the anti-HGF antibody or antigen binding fragment thereof comprises: (i) an immunoglobulin heavy chain comprising the amino acid sequence of SEQ ID NO: 17, or an amino acid sequence having at least 80%, 85%, 90%, 95%, 96%, 97%, 98%, or 99% identity to the amino acid sequence of SEQ ID NO: 17; and (ii) an immunoglobulin light chain comprising the amino acid sequence of SEQ ID NO: 18, or an amino acid sequence having at least 80%, 85%, 90%, 95%, 96%, 97%, 98%, or 99% identity to the amino acid sequence of SEQ ID NO: 18. In certain embodiments, the anti-HGF antibody is selected from the group consisting of: rilotumumab, ficlatuzumab, and combinations thereof, e.g., the anti-HGF antibody is ficlatuzumab.

In certain embodiments, the EGFR inhibitor is an anti-EGFR antibody or antigen binding fragment thereof. In certain embodiments, the anti-EGFR antibody or antigen binding fragment thereof comprises: (i) an immunoglobulin heavy chain variable region comprising a CDRIn comprising the amino acid sequence of SEQ ID NO: 9, a CDR_{H2} comprising the amino acid sequence of SEQ ID NO: 10, and a CDR_{H3} comprising the amino acid sequence of SEQ ID NO: 11; and/or (ii) an immunoglobulin light chain variable region comprising a CDRLA comprising the amino acid sequence of SEQ ID NO: 12, a CDR_{L2} comprising the amino acid sequence of SEQ ID NO: 13, and a CDR_{L3} comprising the amino acid sequence of SEQ ID NO: 14. In certain embodiments, the anti-EGFR antibody or antigen binding fragment thereof comprises: (i) an immunoglobulin heavy chain variable region comprising the amino acid sequence of SEQ ID NO: 15, or an amino acid sequence having at least 80%, 85%, 90%, 95%, 96%, 97%, 98%, or 99% identity to the amino acid sequence of SEQ ID NO: 15; and (ii) an immunoglobulin light chain variable region comprising the amino acid sequence of SEQ ID NO: 16, or an amino acid sequence having at least 80%, 85%, 90%, 95%, 96%, 97%, 98%, or 99% identity to the amino acid sequence of SEQ ID NO: 16. In certain embodiments, the anti-EGFR antibody or antigen binding fragment thereof comprises: (i) an immunoglobulin heavy chain comprising the amino acid sequence of SEQ ID NO: 19, or an amino acid sequence having at least 80%, 85%, 90%, 95%, 96%, 97%, 98%, or 99% identity to the amino acid sequence of SEQ ID NO: 19; and (ii) an immunoglobulin light chain comprising the amino acid sequence of SEQ ID NO: 20, or an amino acid sequence having at least 80%, 85%, 90%, 95%, 96%, 97%, 98%, or 99% identity to the amino acid sequence of SEQ ID NO: 20. In certain embodiments, the anti-EGFR antibody is selected from the group consisting of: cetuximab, futuximab, imgatuzumab, matuzumab, necitumumab, nimotuzumab, panitumumab, amivantamab, zalutumumab, and combinations thereof, e.g., the anti-EGFR antibody is cetuximab.

The anti-HGF antibody or antigen binding fragment thereof or anti-EGFR antibody or antigen binding fragment thereof may, for example, be a Fab, a Fv, a scFv, a Fab′, or a (Fab′)2. In certain embodiments, the anti-HGF antibody or antigen binding fragment thereof or anti-EGFR antibody or antigen binding fragment thereof is, or is derived from, a chimeric antibody, a human antibody, or a humanized antibody.

The HGF inhibitor (e.g., anti-HGF antibody or antigen binding fragment thereof) and EGFR inhibitor (e.g., anti-EGFR antibody or antigen binding fragment thereof) may, for example, be administered concurrently for at least one cycle of treatment. The HGF inhibitor (e.g., anti-HGF antibody or antigen binding fragment thereof) or EGFR inhibitor (e.g., anti-EGFR antibody or antigen binding fragment thereof) may, for example, be administered sequentially for at least one cycle of treatment. In certain embodiments, the HGF inhibitor (e.g., anti-HGF antibody or antigen binding fragment thereof) is administered after the EGFR inhibitor (e.g., anti-EGFR antibody or antigen binding fragment thereof) for at least one cycle of treatment, for example, the HGF inhibitor (e.g., anti-HGF antibody or antigen binding fragment thereof) is administered at least 15 minutes, at least 30 minutes, at least 60 minutes, at least 120 minutes, at least 180 minutes, at least 240 minutes, or at least 300 minutes, or from about 30 minutes to about 60 minutes, from about 60minutes to about 120 minutes, or from about 120 minutes to about 180 minutes, after completion of the administration of the EGFR inhibitor (e.g., anti-EGFR antibody or antigen binding fragment thereof). In certain embodiments, the HGF inhibitor (e.g., anti-HGF antibody or antigen binding fragment thereof) is administered concurrently with the EGFR inhibitor (e.g., anti-EGFR antibody or antigen binding fragment thereof) for at least one cycle of treatment. For example, the HGF inhibitor, e.g., ficlatuzumab, and the EGFR inhibitor, e.g., cetuximab, are administered concurrently, for example, by intravenous infusion.

In certain embodiments, the HGF inhibitor (e.g., anti-HGF antibody or antigen binding fragment thereof) and the EGFR inhibitor (e.g., anti-EGFR antibody or antigen binding fragment thereof) are each administered about every week, about every two weeks, about every three weeks, or about every four weeks. The HGF inhibitor (e.g., anti-HGF antibody or antigen binding fragment thereof) and/or the EGFR inhibitor (e.g., anti-EGFR antibody or antigen binding fragment thereof) may, for example, be administered by intravenous infusion. For example, the HGF inhibitor, e.g., ficlatuzumab, and the EGFR inhibitor, e.g., cetuximab, are administered concurrently, for example, by intravenous infusion every two weeks.

Contemplated doses for the HGF inhibitor (e.g., anti-HGF antibody or antigen binding fragment thereof, such as ficlatuzumab) include about 2 mg/kg, about 5 mg/kg, about 10 mg/kg, about 15 mg/kg, about 20 mg/kg, or about 25 mg/kg. Contemplated doses for the EGFR inhibitor (e.g., anti-EGFR antibody or antigen binding fragment thereof, such as cetuximab) include about 200 mg/m², about 250 mg/m², about 300 mg/m², about 400 mg/m², about 500 mg/m², about 600 mg/m², about 700 mg/m², or about 800 mg/m².

In certain embodiments, the method comprises administering to the subject (i) 20 mg/kg ficlatuzumab (e.g., about every two weeks), and (ii) 500 mg/m² cetuximab (e.g., about every two weeks).

In another aspect, the invention provides a composition comprising (i) an HGF inhibitor (e.g., anti-HGF antibody or antigen binding fragment thereof, e.g., an anti-HGF antibody or antigen binding fragment thereof disclosed herein), (ii) an EGFR inhibitor (e.g., anti-EGFR antibody or antigen binding fragment thereof, e.g., an anti-EGFR antibody or antigen binding fragment thereof disclosed herein), and optionally (iii) a pharmaceutically acceptable carrier.

### BRIEF DESCRIPTION OF THE FIGURES

The invention can be more completely understood with reference to the following figures.

FIG. 1 shows the study protocol for the Phase II trial described in Example 1 to determine the efficacy of treating subjects with recurrent or metastatic HNSCC with ficlatuzumab alone or in combination with cetuximab.

FIG. 2 shows the patient disposition for the Phase II trial described in Example 1 to determine the efficacy of treating subjects with recurrent or metastatic HNSCC with ficlatuzumab alone or in combination with cetuximab, and provides the overall response rate (ORR) for each study arm, including the ORR for the patient population when stratified by HPV status.

FIG. 3 provides a categorization of baseline patient characteristics for subjects participating in the Phase II trial described in Example 1 to determine the efficacy of treating these subjects with recurrent or metastatic HNSCC with ficlatuzumab alone or in combination with cetuximab.

FIG. 4 provides a categorization of the types of toxicities experienced by subjects participating in the Phase II trial described in Example 1 to determine the efficacy of treating these subjects with recurrent or metastatic HNSCC with ficlatuzumab alone or in combination with cetuximab. Toxicities are categorized as cardiovascular, constitutional, dermatologic, gastrointestinal, metabolic or pulmonary, and the percent of each type by Grade 1-2 or Grade 3 are provided, according to treatment type (i.e., ficlatuzumab vs. ficlatuzumab with cetuximab).

FIG. 5A is a Kaplan-Meier curve showing progression free survival (PFS) for the patient population in the Phase II trial described in Example 1 stratified by treatment vs. control arm. The tail of the ficlatuzumab+cetuximab (treatment) arm extends past 15 months. FIG. 5B is a Kaplan-Meier curve showing overall survival data for the patient population in the Phase II trial described in Example 1 stratified by treatment vs. control arm. The tail of the ficlatuzumab+cetuximab arm (treatment) extends past 15 months.

FIG. 6A shows the overall rate of response (ORR) and median Progression Free Survival (mPFS) in the treatment arm (ficlatuzumab+cetuximab) of the Phase II trial described in Example 1 when patient populations are stratified by HPV status (HPV+and HPV-subgroups). (PR=partial response; CR=complete response). FIG. 6B is a Kaplan-Meier curve of the progression free survival (PFS) of the treatment arm (ficlatuzumab+cetuximab) of the Phase II trial described in Example 1 when stratified by HPV status (HPV+and HPV-subgroups). The tail of the HPV-subgroup extends past 15 months.

### DETAILED DESCRIPTION OF THE DISCLOSURE

The present invention relates to improved methods of treating subjects with cancers, such as head and neck squamous cell carcinoma (HNSCC).

Head and neck squamous cell carcinoma (HNSCC) is a morbid and lethal cancer caused by habitual exposure to tobacco and other carcinogens or by the human papillomavirus (HPV). HNSCC is expected to afflict more than 65,000 people in the U.S. and 700,000 people worldwide in 2021. 90% of patients with HPV-positive or HPV-negative HNSCC present with disease localized to the head and neck. Initial treatments for localized disease, referred to as definitive-intent or curative-intent therapies, include surgical resection, radiation and systemic therapy. However, recurrence after curative-intent multimodality treatment is approximately 20% and 50%, respectively. Overall survival in the face of recurrent/metastatic disease is less than two years. Treatments for recurrent/metastatic disease are referred to as palliative therapies. Systemic options for palliative treatment of recurrent/metastatic disease in first line include the anti-programmed death receptor-1 (PD-1) immune checkpoint inhibitor, such as pembrolizumab, with or without platinum and 5-fluorouracil cytotoxic chemotherapy. In second or later line, available therapies include the anti-microtubule taxane chemotherapies, the anti-metabolite methotrexate, and the anti-epidermal growth factor receptor (EGFR) monoclonal antibody (mAb), cetuximab where single agent response rates are on the order of 5-15%.

Head and neck squamous cell carcinomas (HNSCCs) develop from the mucosal epithelium in the oral cavity, pharynx, larynx, and the sinonasal tract (sinuses and nasal cavity) and are the most common malignancies arising in the head and neck. The oral cavity includes the gums, lips, buccal mucosa (lining of the cheeks and back of the lips), hard palate (bony top of the mouth), anterior tongue, floor of the mouth (under the tongue) and retromolar trigone. The pharynx (throat) includes the nasopharynx, oropharynx (palatine tonsils, lingual tonsils, base of the tongue, soft palate, uvula, and posterior pharyngeal wall), and hypopharynx (the bottom part of the throat extending from the hyoid bone to the cricoid cartilage) and the larynx. HNSCC may develop at any of these primary sites. HNSCC may also develop in the salivary glands. HNSCC tumors arising at any of these sites may be treated according to methods of the invention disclosed herein. HNSCC includes, for example, hypopharyngeal cancer, laryngeal cancer, lip and oral cavity cancer, nasopharyngeal cancer, oropharyngeal cancer, paranasal sinus and nasal cavity cancer, and salivary gland cancer. These are exemplary types of HNSCC that may be treated according to methods of the invention disclosed herein. However, squamous cell carcinomas of unknown primary origin that are clearly related to the head and neck may be treated according to the methods of the invention.

HNSCC tumors are typically caused by prior infection with oncogenic strains of human papillomavirus (HPV) such as HPV-16 or HPV-18, as well as other strains, or smoking or tobacco use. HPV negative HNSCCs of the oral cavity and the larynx are primarily caused by smoking or tobacco use. HNSCCs caused by HPV (HPV-positive) typically arise from the palatine and lingual tonsils of the oropharynx, whereas HNSCCs associated with tobacco use are primarily found in the oral cavity, hypopharynx and larynx.

A key finding of the Phase II study described in Example 1 herein below is that HNSCC subjects whose tumors were HPV-negative had superior outcomes from treatment with ficlatuzumab in combination with cetuximab as compared to subjects with HPV-positive HNSCC. In fact, as the data provided in Example 1 shows, there were no responders in the combination arm (ficlatuzumab with cetuximab) among HPV-positive HNSCC subjects; all responders (complete and partial response) had HPV-negative HNSCC. In the ficlatuzumab only arm, the only responder was HPV-negative; there were no responders that were HPV-positive. This outcome was surprising as, historically, HPV-positive HNSCC subjects typically have better outcomes than HPV-negative HNSCC subjects. In fact, being HPV-negative is considered a poor prognostic indicator for HNSCC patients, including those being treated with cetuximab according to the current standard of care in second or later line therapies. Accordingly, that HPV-negative status for treatment according to the methods of the invention would be a positive prognostic indicator, whereas HPV-positive status would be a negative prognostic indicator is entirely surprising and unexpected.

Accordingly, among other things, the data presented herein suggest that HPV-negative HNSCC subjects should be selected for treatment with ficlatuzumab and cetuximab, whereas subjects with HPV-positive HNSCC should not be selected for treatment with ficlatuzumab and cetuximab. Further, the data suggest that cetuximab with ficlatuzumab could replace cetuximab alone as the current standard of care for second line therapy or later line therapy in subjects that have HPV-negative HNSCC. For example, in HNSCC subjects that have progressed after treatment with immunotherapy (e.g., PD-1, PD-L1, or other checkpoint inhibitors) or other first line treatments that are the standard of care, if their HNSCC is HPV−, they could be treated with ficlatuzumab and cetuximab according to the methods of the invention disclosed herein, rather than being treated with cetuximab only. Accordingly, according to some embodiments of the invention, subjects treated with ficlatuzumab and cetuximab according to the methods of the invention are cetuximab naïve, meaning they have not been treated previously with cetuximab. In some embodiments of the invention, HNSCC subjects treated with ficlatuzumab and cetuximab according to the methods of the invention are cetuximab-resistant, meaning their HNSCC has been treated previously with cetuximab but the HNSCC recurred or metastasized.

According to one embodiment of the invention, a subject with HNSCC is treated according to the methods of the invention (e.g., with ficlatuzumab and cetuximab) if they are identified as having HPV-negative HNSCC. In one embodiment, the subject is cetuximab naïve.

According to one embodiment of the invention, a subject with recurrent or metastatic HNSCC is treated according to the methods of the invention (e.g., with ficlatuzumab and cetuximab) if they are identified as having HPV-negative HNSCC. In one embodiment, the subject is cetuximab naïve.

According to some embodiments of the invention, a subject with HNSCC is treated according to the methods of the invention (e.g., with ficlatuzumab and cetuximab) if they are identified as having HPV-negative HNSCC and the HNSCC is resistant to treatment with immunotherapy (e.g., immune checkpoint inhibitor therapy, such as pembrolizumab), for example, the HNSCC is immune checkpoint inhibitor-, PD-L1-, or PD-1-resistant. In some embodiments, the subject may have also been previously treated with radiation. In one embodiment, the subject is cetuximab naïve. In some embodiments, the HNSCC has not previously been treated with a platinum and is therefore platinum-naïve, or the subject is platinum-ineligible.

According to some embodiments of the invention, a subject with HNSCC is treated according to the methods of the invention (e.g., with ficlatuzumab and cetuximab) if they are identified as having HPV-negative HNSCC and the HNSCC is resistant to immunotherapy (e.g., immune checkpoint inhibitor therapy, such as pembrolizumab) and the HNSCC has been treated with a platinum chemotherapy (e.g., cisplatin or carboplatin). In some embodiments, the subject may have also been previously treated with radiation. In some embodiments, the HNSCC is also platinum-resistant. In one embodiment, the subject is cetuximab naïve.

According to one embodiment of the invention, a subject with recurrent or metastatic HNSCC is treated according to the methods of the invention (e.g., with ficlatuzumab and cetuximab) if they are identified as having HPV-negative HNSCC and the HNSCC is resistant to treatment with immunotherapy (e.g., immune checkpoint inhibitor therapy, such as pembrolizumab), for example, the HNSCC is immune checkpoint inhibitor-, PD-L1-, or PD-1-resistant. In some embodiments, the subject may have also been previously treated with radiation. In one embodiment, the subject is cetuximab naïve. In some embodiments, the HNSCC has not previously been treated with a platinum and is therefore platinum-naïve, or the subject is platinum-ineligible.

According to one embodiment of the invention, a subject with recurrent or metastatic HNSCC is treated according to the methods of the invention (e.g., with ficlatuzumab and cetuximab) if they are identified as having HPV-negative HNSCC and the HNSCC is resistant to immunotherapy (e.g., immune checkpoint inhibitor therapy, such as pembrolizumab) and the HNSCC has been treated with a platinum chemotherapy (e.g, cisplatin or carboplatin). In some embodiments, the subject may have also been previously treated with radiation. In some embodiments, the HNSCC is also platinum-resistant. In one embodiment, the subject is cetuximab naïve.

According to one embodiment of the invention, a subject with recurrent or metastatic HNSCC is treated according to the methods of the invention (e.g., with ficlatuzumab and cetuximab) if they are identified as having HPV-negative HNSCC and the HNSCC is platinum resistant. In some embodiments, the subject may have also been previously treated with radiation. In some embodiments, the subject is immunotherapy ineligible (e.g., immune checkpoint inhibitor therapy, such as pembrolizumab), or is immunotherapy-naive (e.g., has not previously received immunotherapy, e.g., immune checkpoint inhibitor therapy, such as pembrolizumab, e.g., is immune checkpoint or PD-1 or PD-L1 naïve). In one embodiment, the subject is cetuximab naïve.

According to one embodiment of the invention, a subject with recurrent or metastatic HNSCC is treated according to the methods of the invention (e.g., with ficlatuzumab and cetuximab) if they are identified as having HPV-negative HNSCC and the HNSCC is platinum resistant. In some embodiments, the subject may have also been previously treated with radiation. In some embodiments, the HNSCC may also be immunotherapy-resistant (e.g., immune checkpoint inhibitor therapy, such as pembrolizumab), or the subject was treated with immunotherapy (e.g., an immune checkpoint inhibitor therapy, such as pembrolizumab). In some embodiments, the subject is immunotherapy ineligible (e.g., immune checkpoint inhibitor therapy, such as PD-1 or PD-L1 ineligible), or the subject may not have been previously treated with immunotherapy and is therefore immunotherapy-naive. In one embodiment, the subject is cetuximab naïve.

According to one embodiment of the invention, a subject with HNSCC, for example, recurrent or metastatic HNSCC, is treated according to the methods of the invention (e.g., with ficlatuzumab and cetuximab) if they are identified as having HPV-negative HNSCC and the HNSCC is platinum resistant and the HNSCC is immunotherapy-resistant. In one embodiment, the subject is cetuximab naïve. In some embodiments, a subject with HNSCC, for example, recurrent or metastatic HNSCC, is treated according to the methods of the invention (e.g., with ficlatuzumab and cetuximab) if they are identified as having HPV-negative HNSCC and the HNSCC is platinum resistant and/or the HNSCC is immunotherapy-resistant. In some embodiments, an immunotherapy comprises an immune checkpoint inhibitor therapy as described herein. In some embodiments, the HNSCC may also have been treated with radiation.

In some embodiments, an HNSCC that is platinum resistant is resistant to carboplatin, oxaliplatin, cisplatin, nedaplatin, lobaplatin, triplatin tetranitrate, phenanthriplatin, picoplatin, or satraplatin, or combinations thereof. In some embodiments, an HNSCC that is platinum resistant is carboplatin or cisplatin, or combinations thereof.

In some embodiments, an HNSCC that is immunotherapy resistant is resistant to a checkpoint inhibitor. In some embodiments, an immune checkpoint inhibitor may include a PD-1 inhibitor, a PD-L1 inhibitor, a CTLA-4 inhibitor, a TIM-3 inhibitor, a LAG-3 inhibitor, a TIGIT inhibitor, a VISTA inhibitor, a KIR inhibitor, a 2B4 inhibitor, a CD160 inhibitor, a CGEN-15049 inhibitor, a CHK1 inhibitor, a CHK2 inhibitor, a A2aR inhibitor, or any combination thereof.

In some embodiments, a PD-1 inhibitor may include nivolumab, pembrolizumab, pidilizumab, REGN2810, PDR001, or any combination thereof. In some embodiments, a PD-L1 inhibitor may include durvalumab, atezolizumab, avelumab, or any combination thereof. In some embodiments, a CTLA-4 inhibitor may include ipilimumab, tremelimumab, AGEN-1884, or any combination thereof. In some embodiments, a TIM-3 inhibitor may include TSR-022, LY3321367, MBG453, or any combination thereof. In some embodiments, a TIGIT inhibitor may include BMS-986207, AGEN17, tiragolumab, MK-7684, OMP-313M32, EOS-448, AB154, or combinations thereof. In some embodiments, a LAG-3 inhibitor may include BMS-986016, REGN3767, IMP321, LAG525, BI754111, favezelimab, or combinations thereof. In some embodiments, a VISTA inhibitor may include CI-8993, HMBD-002, a PSGL-1 antagonist as described in WO 2018/132476, or combinations thereof.

In one aspect, the invention provides a method of treating cancer in a subject in need thereof. The method comprises administering to the subject a therapeutically effective amount of an HGF inhibitor and an EGFR inhibitor. In another aspect, the invention provides a composition comprising an HGF inhibitor, an EGFR inhibitor, and optionally a pharmaceutically acceptable carrier. In another aspect, the invention provides a method of identifying a subject with HNSCC that is suitable for a combination therapy comprising an HGF inhibitor and an EGFR inhibitor.

### I. Definitions

For convenience, certain terms in the specification, examples, and appended claims are collected in this section.

As used herein, “pharmaceutically acceptable” or “pharmacologically acceptable” mean molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein, the term “Progression-Free Survival (PFS)” is defined as the time from randomization to first documentation of objective tumor progression (progressive disease “PD”, radiological) according to RECIST (Version 1.1; see, e.g., Eisenhauer et al. (2009), EUR. J. CANCER, 25:228-247) or death due to any reason, whichever comes first.

As used herein, the term “Overall survival (OS)” is defined as the time from the date of randomization to date of death due to any cause.

As used herein, the term “Objective response rate (ORR)” is defined as the proportion of subjects with confirmed complete response (CR) or confirmed partial response (PR) according to RECIST (Version 1.1), relative to the total population of randomized subjects. Confirmed responses are those that persist on repeat imaging study at least 4 weeks after the initial documentation of response.

As used herein, the term “Duration of response (DoR)” is defined as the time from the first documentation of objective tumor response (either complete or partial, whichever is recorded first) to the first documentation of objective tumor progression or to death due to any cause.

As used herein, the terms “response” or “responding” in the context of a subject's response to a treatment refer to the RECIST (Response Evaluation Criteria in Solid Tumors, version 1.1, 2009) criteria for evaluating response of target lesions to a cancer therapy. According to the RECIST criteria, subjects who respond are categorized as either “complete responders” (disappearance of all target lesions; any pathological lymph nodes (whether target or non-target) must have reduction in short axis to <10 mm) or “partial responders” (at least a 30% decrease in the sum of the longest diameter of target lesions, taking a reference the baseline sum longest diameter); non-responders are placed into one of two categories: stable disease (neither sufficient shrinkage to qualify for partial response nor sufficient increase to qualify for progressive disease, taking as reference the smallest sum longest diameter since start of treatment) or progressive disease (at least a 20% increase in the sum of the longest diameter of target lesions, taking as reference the smallest sum longest diameter recorded since treatment started or the appearance of one or more new lesions; the baseline sum diameter measurements; in addition to the relative increase of 20%, the sum must also demonstrate an absolute increase of least 5 mm). The RECIST criteria are discussed in detail in, e.g., Therasse et al., J. NATL. CANCER INST., 2000:92:205-216 (RECIST 1.0), and Eisenhauer et al., EUR. J. CANCER, 2009:25:228-247 (RECIST 1.1). Accordingly, as described herein, responding to therapy refers to subjects falling within the RECIST categories of complete or partial responder, whereas not responding refers to subjects falling within the RECIST categories of stable disease or progressive disease.

As used herein, the term “drug related adverse event,” “adverse event” or “AE” refers to adverse events as defined and classified in the National Cancer Institute-Common Terminology Criteria for Adverse Events (CTCAE) version 4.03 dated Jun. 14, 2010, and any reference to “Grade” of adverse event refers to the grading system as outlined therein.

As used herein, the terms “treating” or “treat” or “treatment” in the context of cancer refer to applying techniques, actions or therapies to a subject that (a) slow tumor growth, (b) halt tumor growth, (c) promote tumor regression or disappearance, (d) ameliorate a symptom of the cancer, (e) cure the cancer, or (f) prolong survival of the subject, or applying techniques, actions or therapies to a subject in an attempt to achieve any of (a)-(f) regardless of whether the individual actually responds to the technique, action or therapy.

As used herein, the term “clinical benefit” refers to a subject experiencing any of (a) slowing of tumor growth, (b) halting of tumor growth, (c) tumor regression or disappearance, (d) amelioration of a symptom of the cancer, (e) curing the cancer, or (f) prolonging survival of the subject.

As used herein, “advanced” with respect to a cancer or tumor (e.g., HNSCC) refers to cancer or tumor that has reached Stage 3 or Stage 4. In certain embodiments, “advanced” means that the cancer or tumor has metastasized, or otherwise cannot be adequately treated with local therapy, such as surgical intervention or radiation therapy, alone, and therefore requires a systemic therapy. In certain embodiments, “advanced” means that the cancer or tumor has recurred after having responded to treatment with a local or systemic therapy.

As used herein, “pan-refractory” HNSCC refers to a HNSCC that is resistant to, or ineligible for, treatment with each of (i) platinum chemotherapy, (ii) an immunotherapy (e.g., a checkpoint inhibitor), and (iii) cetuximab.

As used herein, “recurrent” cancer (e.g., recurrent HNSCC) refers to a cancer that fails to respond to treatment or returns after treatment, i.e., “recurs.” For example, a cancer is recurrent if it fails to respond to a mode of treatment, e.g., the subject fails to attain a clinical benefit, or experiences disease progression while undergoing treatment. For example, a cancer is recurrent if it returns or progresses after treatment. As used herein, “recurrent” cancer may be a cancer that responds to an initial treatment, and then returns, or is a cancer that initially responds to a treatment but later in the treatment stops responding or develops resistance to such treatment. In certain embodiments, “recurrent” refers to a cancer or tumor, such as a HNSCC, that has been treated with at least one systemic or local treatment, and has not responded to such treatment or becomes resistant to such treatment, or that continues to progress during or after such treatment. In another embodiment, “recurrent” refers to a cancer or tumor, such as a HNSCC, that has been treated with at least two systemic or local treatments, and has not responded to such treatment or becomes resistant to such treatment, or that continues to progress during or after such treatment. In some embodiments, HNSCC is “recurrent” if it fails to respond to definitive-intent or curative-intent therapy and requires palliative therapy. In some scientific contexts, an HNSCC that is recurrent can be described as “resistant” or “refractory”

As used herein, “metastatic” cancer (e.g., metastatic HNSCC) refers to a cancer that has spread from the part of the body where it started (i.e., the primary site) to another part of the body. For example, metastatic HNSCC refers to HNSCC primary site tumors that have spread to other parts of the body. In some embodiments, metastatic HNSCC requires palliative therapy.

As used herein, the terms “subject” and “patient” are used interchangeably and refer to an organism to be treated by the methods and compositions of the present invention. Such organisms are preferably a mammal (e.g., human, mouse, rat, guinea pig, dog, cat, horse, cow, pig, or non-human primate, such as a monkey, chimpanzee, baboon, and rhesus), and more preferably, a human.

As used herein, the term “pharmaceutical composition” refers to the combination of an active agent with a carrier, inert or active, making the composition especially suitable for diagnostic or therapeutic use in vivo or ex vivo.

As used herein, the term “effective amount” refers to the amount of an active agent (e.g., an HGF inhibitor and/or EGFR inhibitor) sufficient to effect beneficial or desired results, such as, for example, to effect a clinical benefit in a subject. An effective amount can be administered in one or more administrations, applications or dosages and is not intended to be limited to a particular formulation or administration route.

As used herein, the term “resistant” refers to a cancer or tumor that does not respond to a type of anti-cancer therapy. In some embodiments, a cancer or tumor is “resistant” if it previously responded to a therapy and then stops responding to the therapy at some point during a course of treatment, or the cancer or tumor may be “resistant” because it never responds to a course of therapy, e.g., it is non-responsive to a therapy from the beginning of a course of treatment. In some embodiments, a cancer or tumor is also “resistant” if the cancer or tumor recurs or progresses after a course of treatment is completed. In some embodiments, a cancer or tumor is also “resistant” if the cancer or tumor recurs or progresses during a course of treatment. For example, a “platinum-resistant” cancer or tumor, e.g., an HNSCC, is one that does not respond or stops responding to a platinum chemotherapy, e.g., carboplatin or cisplatin. For example, an immunotherapy-resistant cancer or tumor, e.g., an HNSCC, is one that does not respond or stops responding to an immunotherapy, e.g., a checkpoint inhibitor, such as a PD-1 or PD-L1 inhibitor, such pembrolizumab. For example, an HNSCC that does not respond or stops responding to a checkpoint inhibitor is a “checkpoint inhibitor-resistant” HNSCC. In addition, a cancer of tumor, such as an HNSCC, that “fails” immunotherapy or is “non-responsive” to immunotherapy can be synonymous with an HNSCC that is “immunotherapy-resistant.” A cancer or tumor, such as an HNSCC, that “fails” platinum or is “non-responsive” to platinum chemotherapy is considered to be synonymous with an HNSCC that is “platinum-resistant.”

As used herein, the term “platinum-ineligible” refers to a subject who is not eligible to receive a platinum chemotherapy, as a therapy for cancer, such as HNSCC, for any reason. For example, a subject may be platinum-ineligible due to renal or hepatic dysfunction, or due to cumulative toxicities.

As used herein, the term “immunotherapy-ineligible” refers to a subject who is not eligible to receive an immunotherapy, such as a checkpoint inhibitor, e.g., a PD-1 or PD-L1 inhibitor, as therapy for cancer, such as HNSCC, for any reason. For example, a subject may be immunotherapy-ineligible because the subject has an autoimmune disorder.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred. For example, “about 10” is a disclosure of the value 10, as well as the range of 10±10%.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, singular forms “a,” “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5 fold, etc., 2.1, 2.2, 2.3, 2.4, 2.5 fold, etc., and so forth.

### II. HGF Inhibitors

### 1) HGF/c-Met in Cancer

The MET oncogene encodes c-Met, an RTK bound exclusively by the ligand, hepatocyte growth factor (HGF). HGF is also known as “scatter factor;” this designation arose from early observations that HGF stimulates cellular decoupling and motogenesis. Overexpression of c-Met is transformative for normal cells and enhances motility, invasion/metastasis and angiogenesis (Peruzzi, B. and Bottaro, D. P. Clin Cancer Res. (2006) 12:3657-3660). MET is an established driver of epithelial-to-mesenchymal transition.

c-Met and/or HGF are overexpressed in ˜80% of HNSCC (Knowles, L. M. et al. n Cancer Res. (2009) 15:3740-3750), and MET amplification has been reported in 13% of HNSCC tumors (Seiwert, T. Y. et al. Cancer Res. (2009) 69:3021-3031). Moreover, several mutations have been identified in the MET oncogene in HNSCC, including alterations in the semaphorin ligand-binding, juxtamembrane, and RTK domains (Seiwert, T. Y. et al. Cancer Res. (2009) 69:3021-3031). An activating point mutation (Y1253D) was described in 14% of patients in a Swiss chemoradiotherapy trial for locally advanced disease and predicted decreased metastasis-free survival, although the presence of this mutation in HNSCC was not confirmed in two whole-exome sequencing projects (Ghadjar, P. et al. Clin Exp Metastasis. (2009) 26:809-815; Stransky, N. et al. Science (2011) 333:1157-1160; Agrawal, N. et al. Science (2011) 333:1154-1157).

### 2) HGF Inhibitors

It is contemplated that a variety of HGF inhibitors can be used in the practice of the invention. The inhibitors can completely or partially inhibit or otherwise reduce a given HGF activity or a given HGF mediated activity relative to an untreated control sample (e.g., a tissue or body fluid sample) or subject. For example, certain HGF inhibitors may act by blocking, reducing or otherwise neutralizing binding between HGF and an HGF ligand (e.g., c-Met). It is understood that that an HGF inhibitor may block, reduce, or otherwise neutralize binding between HGF and an HGF ligand by binding, directly or indirectly, to HGF, or alternatively, by binding, directly or indirectly, to the HGF ligand (e.g., c-Met). Alternatively or in addition, the HGF inhibitor may act by reducing the expression of HGF or an HGF ligand (e.g., c-Met). Alternatively or in addition, the HGF inhibitor, directly or indirectly, may inhibit the downstream effects of the interaction between HGF and an HGF ligand (e.g. c-Met).

Exemplary HGF inhibitors include antibodies, nucleic acid-based therapeutics, such as aptamers and spiegelmers that bind to a target of interest, such as HGF, or antisense or siRNA molecules or CRISPR systems that inhibit expression and/or activity of a target of interest, such as HGF, or small molecule inhibitors, for example, small molecule inhibitors of HGF, or a combination thereof.

It is understood that, in certain embodiments, different HGF inhibitors may be administered in combination.

### 3) Anti-HGF Antibodies

In certain embodiments, the HGF inhibitor is an anti-HGF antibody or antigen binding fragment thereof.

In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody.

Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

As disclosed herein, antibodies of the invention may comprise: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and/or the light chain variable region together define a single binding site for binding HGF.

In certain embodiments, an anti-HGF antibody can comprise: an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3, wherein CDR_{H1}, CDR_{H2}, and CDR_{H3} sequences are interposed between immunoglobulin FR sequences; and/or an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6, wherein the CDR_{L1}, CDR_{L2}, and CDR_{L3} sequences are interposed between immunoglobulin FR sequences.

In certain embodiments, an anti-HGF antibody can comprise: an immunoglobulin heavy chain variable region comprising a CDR_{H1} comprising an amino acid sequence having at least 80%, 85%, 90%, 95%, 96%, 97%, 98%, or 99% identity to the amino acid sequence of SEQ ID NO: 1, a CDR_{H2} comprising an amino acid sequence having at least 80%, 85%, 90%, 95%, 96%, 97%, 98%, or 99% identity to the amino acid sequence of SEQ ID NO: 2, and a CDR_{H3} comprising an amino acid sequence having at least 80%, 85%, 90%, 95%, 96%, 97%, 98%, or 99% identity to the amino acid sequence of SEQ ID NO: 3, wherein CDR_{H1}, CDR_{H2}, and CDR_{H3} sequences are interposed between immunoglobulin FR sequences; and/or an immunoglobulin light chain variable region comprising a CDR_{L1} comprising an amino acid sequence having at least 80%, 85%, 90%, 95%, 96%, 97%, 98%, or 99% identity to the amino acid sequence of SEQ ID NO: 4, a CDR_{L2} comprising an amino acid sequence having at least 80%, 85%, 90%, 95%, 96%, 97%, 98%, or 99% identity to the amino acid sequence of SEQ ID NO: 5, and a CDR_{L3} comprising an amino acid sequence having at least 80%, 85%, 90%, 95%, 96%, 97%, 98%, or 99% identity to the amino acid sequence of SEQ ID NO: 6, wherein the CDR_{L1}, CDR_{L2}, and CDR_{L3} sequences are interposed between immunoglobulin FR sequences.

In certain embodiments, the anti-HGF antibody comprises an immunoglobulin heavy chain variable region comprising the amino acid sequence of SEQ ID NO: 7, and an immunoglobulin light chain variable region comprising the amino acid sequence of SEQ ID NO: 8.

In certain embodiments, the anti-HGF antibody comprises an immunoglobulin heavy chain variable region comprising an amino acid sequence that is at least 70%, 75%, 80%, 85%, 90%, 95%, 96%, 97%, 98%, or 99% identical to the entire variable region and/or the framework region sequence of the amino acid sequence of SEQ ID NO: 7. In certain embodiments, the anti-HGF antibody comprises an immunoglobulin light chain variable region comprising an amino acid sequence that is at least 70%, 75%, 80%, 85%, 90%, 95%, 96%, 97%, 98%, or 99% identical to the entire variable region and/or the framework region sequence of the amino acid sequence of SEQ ID NO: 8.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that bind HGF may contain amino acid alterations, e.g., at least 1, 2, 3, 4, 5, or 10 amino acid substitutions, deletions, or additions, e.g., in the framework regions of the heavy and/or light chain variable regions.

In certain embodiments, it is contemplated that a heavy chain variable region sequence, for example, the V_{H} sequence of SEQ ID NO: 7, or any variants thereof, may be covalently linked to a variety of heavy chain constant region sequences known in the art. Similarly, it is contemplated that a light chain variable region sequence, for example, the V_{L} of SEQ ID NO: 8, or any variants thereof, may be covalently linked to a variety of light chain constant region sequences known in the art.

For example, the antibody molecule may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and/or can fix complement. In other embodiments the antibody does not recruit effector cells and/or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the anti-HGF antibody comprises an immunoglobulin heavy chain comprising the amino acid sequence of SEQ ID NO: 17, or an amino acid sequence that has at least 85%, 90%, 95%, 96%, 97%, 98%, or 99% sequence identity to SEQ ID NO: 17; and/or an immunoglobulin light chain comprising the amino acid sequence of SEQ ID NO: 18, or an amino acid sequence that has at least 85%, 90%, 95%, 96%, 97%, 98%, or 99% sequence identity to SEQ ID NO: 18.

In certain embodiments, the antibody binds human HGF with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, 0.05 nM, 0.01 nM, 0.0075 nM, 0.005 nM, 0.001 nM, or lower, as measured using standard binding assays, for example, surface plasmon resonance or bio-layer interferometry. In certain embodiments, the antibody binds human HGF with a K_{D} of from about 20 nM to about 0.001 nM, from about 20 nM to about 0.005 nM, from about 20 nM to about 0.0075 nM, from about 20 nM to about 0.01 nM, from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.001 nM, from about 10 nM to about 0.005 nM, from about 10 nM to about 0.0075 nM, from about 10 nM to about 0.01 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.001 nM, from about 5 nM to about 0.005 nM, from about 5 nM to about 0.0075 nM, from about 5 nM to about 0.01 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.001 nM, from about 3 nM to about 0.005 nM, from about 3 nM to about 0.0075 nM, from about 3 nM to about 0.01 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.001 nM, from about 2 nM to about 0.005 nM, from about 2 nM to about 0.0075 nM, from about 2 nM to about 0.01 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.001 nM, from about 1 nM to about 0.005 nM, from about 1 nM to about 0.0075 nM, from about 1 nM to about 0.01 nM from about 1 nM to about 0.05 nM, from about 1 nM to about 0.075 nM, from about 1 nM to about 0.1 nM, from about 1 nM to about 0.5 nM, from about 0.5 nM to about 0.001 nM, from about 0.5 nM to about 0.005 nM, from about 0.5 nM to about 0.0075 nM, from about 0.5 nM to about 0.01 nM, from about 0.5 nM to about 0.05 nM, from about 0.5 nM to about 0.075 nM, from about 0.5 nM to about 0.1 nM, from about 0.1 nM to about 0.001 nM, from about 0.1 nM to about 0.005 nM, from about 0.1 nM to about 0.0075 nM, from about 0.1 nM to about 0.01 nM, from about 0.1 nM to about 0.05 nM, from about 0.1 nM to about 0.075 nM, from about 0.075 nM to about 0.001 nM, from about 0.075 nM to about 0.005 nM, from about 0.075 nM to about 0.0075 nM, from about 0.075 nM to about 0.01 nM, from about 0.075 nM to about 0.05 nM, or from about 0.05 nM to about 0.035 nM, as measured using standard binding assays, for example, surface plasmon resonance or bio-layer interferometry.

In certain embodiments, the invention provides antibodies that bind to the same epitope present in HGF as that bound by a disclosed antibody. In certain embodiments, the invention provides antibodies that compete for binding to HGF with a disclosed antibody.

Competition assays for determining whether an antibody binds to the same epitope as, or competes for binding with a disclosed antibody are known in the art. Exemplary competition assays include immunoassays (e.g., ELISA assays, RIA assays), surface plasmon resonance, (e.g., BlAcore analysis), bio-layer interferometry, and flow cytometry.

Typically, a competition assay involves the use of an antigen (e.g., a human HGF protein or fragment thereof) bound to a solid surface or expressed on a cell surface, a test HGF-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 7, and an immunoglobulin light chain variable region comprising an amino acid sequence that is at least 70%, 75%, 80%, 85%, 90%, 95%, 96%, 97%, 98%, or 99% identical to SEQ ID NO: 8, and (ii) competes for binding to human HGF with and/or binds to same epitope on human HGF as an antibody comprising an immunoglobulin heavy chain variable region comprising the amino acid sequence of SEQ ID NO: 7, and an immunoglobulin light chain variable region comprising the amino acid sequence of SEQ ID NO: 8.

Additional anti-HGF antibodies are described in International (PCT) Patent Application Publication No. WO 2007/143098, the contents of which are fully incorporated by reference.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

In certain embodiments, disclosed antibodies can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

In certain embodiments, the anti-HGF antibody is ficlatuzumab (AV-299). Ficlatuzumab is a humanized HGF inhibitory immunoglobulin G1 (IgG1) monoclonal antibody. The amino acid sequence of the CDR_{H1}, CDR_{H2}, and CDR_{H3} sequences of ficlatuzumab are depicted in SEQ ID NO: 1, 2, and 3, respectively. The amino acid sequence of the CDR_{L1}, CDR_{L2}, and CDR_{L3} sequences of ficlatuzumab are depicted in SEQ ID NO: 4, 5, and 6, respectively. The amino acid sequence of the heavy chain variable region of ficlatuzumab is depicted in SEQ ID NO: 7, and the amino acid sequence of the light chain variable region is depicted in SEQ ID NO: 8. The amino acid sequence of the heavy chain of ficlatuzumab is depicted in SEQ ID NO: 17, and the amino acid sequence of the light chain is depicted in SEQ ID NO: 18.

In certain embodiments, the anti-HGF antibody is rilotumumab. The amino acid sequence of the CDR_{H1}, CDR_{H2}, and CDR_{H3} sequences of rilotumumab are depicted in SEQ ID NO: 21, 22, and 23, respectively. The amino acid sequence of the CDR_{L1}, CDR_{L2}, and CDR_{L3} sequences of rilotumumab are depicted in SEQ ID NO: 24, 25, and 26, respectively. The amino acid sequence of the heavy chain variable region of rilotumumab is depicted in SEQ ID NO: 27,and the amino acid sequence of the light chain variable region is depicted in SEQ ID NO: 28. The amino acid sequence of the heavy chain of rilotumumab is depicted in SEQ ID NO: 29, and the amino acid sequence of the light chain is depicted in SEQ ID NO: 30.

### III. EGFR Inhibitors

### 1) Epidermal Growth Factor Receptor in Cancer

EGFR is a member of the ErbB/HER family of transmembrane glycoprotein receptor tyrosine kinases (RTK). Activated EGFR initiates a pleiotropic network of downstream signaling cascades including Ras/Raf/MAPK, PI3K/Akt, STAT, and Src Kinase effecting cellular proliferation, invasion, angiogenesis and metastasis (Ciardiello, F. et al. European Journal of Cancer (2003) 39:1348-1354).

In vitro, forced overexpression of EGFR causes malignant transformation of oral epithelial cells, suggesting its role as an oncogene in HNSCC. EGFR overexpression as measured by immunohistochemistry (IHC) and increased EGFR gene copy number as measured by fluorescence in situ hybridization occur in the majority of HNSCC, and is associated with increased stage as well as reduced relapse-free and overall survival (OS) (Chung, C. H. et al. J Clin Oncol. (2006) 24:4170-4176; Grandis, J. R. et al. Cancer (1996) 78:1284-1292; Dassonville, O. et al. Journal of Clinical Oncology (1993) 11:1873-1878; Chung, C. H. et al. Int J Radiat Oncol Biol Phys. (2011) 81:331-338).

### 2) EGFR Inhibitors

It is contemplated that a variety of EGFR inhibitors can be used in the practice of the invention. The inhibitors can completely or partially inhibit or otherwise reduce a given EGFR activity or a given EGFR mediated activity relative to an untreated control sample (e.g., a tissue or body fluid sample) or subject. For example, certain EGFR inhibitors may act by blocking, reducing or otherwise neutralizing binding between EGFR and an EGFR ligand (e.g., EGF). It is understood that that an EGFR inhibitor may block, reduce or otherwise neutralize binding between EGFR and an EGFR ligand by binding, directly or indirectly, to EGFR, or alternatively, by binding, directly or indirectly, to the EGFR ligand (e.g., EGF). Alternatively or in addition, the EGFR inhibitor may act by reducing the expression of EGFR or an EGFR ligand (e.g., EGF). Alternatively or in addition, the EGFR inhibitor, directly or indirectly, may inhibit the downstream effects of the interaction between EGFR and an EGFR ligand (e.g. EGF).

Exemplary EGFR inhibitors include antibodies, nucleic acid-based therapeutics, such as aptamers and spiegelmers that bind to a target of interest, such as EGFR, or antisense or siRNA molecules or CRISPR systems that inhibit expression and/or activity of a target of interest, such as EGFR, or small molecule inhibitors, for example, small molecule inhibitors of EGFR, or a combination thereof.

It is understood that, in certain embodiments, different EGFR inhibitors may be administered in combination.

### 3) Anti-EGFR Antibodies

In certain embodiments, the EGFR inhibitor is an anti-EGFR antibody or antigen binding fragment thereof.

In certain embodiments, an anti-EGFR antibody can comprise: an immunoglobulin heavy chain variable region comprising a CDRIll comprising the amino acid sequence of SEQ ID NO: 9, a CDR_{H2} comprising the amino acid sequence of SEQ ID NO: 10, and a CDR_{H3} comprising the amino acid sequence of SEQ ID NO: 11, wherein CDR_{H1}, CDR_{H2}, and CDR_{H3} sequences are interposed between immunoglobulin FR sequences; and/or an immunoglobulin light chain variable region comprising a CDR_{L1} comprising the amino acid sequence of SEQ ID NO: 12, a CDR_{L2} comprising the amino acid sequence of SEQ ID NO: 13, and a CDR_{L3} comprising the amino acid sequence of SEQ ID NO: 14, wherein the CDR_{L1}, CDR_{L2}, and CDR_{L3} sequences are interposed between immunoglobulin FR sequences.

In certain embodiments, an anti-EGFR antibody can comprise: an immunoglobulin heavy chain variable region comprising a CDRIll comprising an amino acid sequence having at least 80%, 85%, 90%, 95%, 96%, 97%, 98%, or 99% identity to the amino acid sequence of SEQ ID NO: 9, a CDR_{H2} comprising an amino acid sequence having at least 80%, 85%, 90%, 95%, 96%, 97%, 98%, or 99% identity to the amino acid sequence of SEQ ID NO: 10, and a CDR_{H3} comprising an amino acid sequence having at least 80%, 85%, 90%, 95%, 96%, 97%, 98%, or 99% identity to the amino acid sequence of SEQ ID NO: 11, wherein CDR_{H1}, CDR_{H2}, and CDR_{H3} sequences are interposed between immunoglobulin FR sequences; and/or an immunoglobulin light chain variable region comprising a CDR_{L1} comprising an amino acid sequence having at least 80%, 85%, 90%, 95%, 96%, 97%, 98%, or 99% identity to the amino acid sequence of SEQ ID NO: 12, a CDR_{L2} comprising an amino acid sequence having at least 80%, 85%, 90%, 95%, 96%, 97%, 98%, or 99% identity to the amino acid sequence of SEQ ID NO: 13, and a CDR_{L3} comprising an amino acid sequence having at least 80%, 85%, 90%, 95%, 96%, 97%, 98%, or 99% identity to the amino acid sequence of SEQ ID NO: 14, wherein the CDR_{L1}, CDR_{L2}, and CDR_{L3} sequences are interposed between immunoglobulin FR sequences.

In certain embodiments, the anti-EGFR antibody comprises an immunoglobulin heavy chain variable region comprising the amino acid sequence of SEQ ID NO: 15, and an immunoglobulin light chain variable region comprising the amino acid sequence of SEQ ID NO: 16.

In certain embodiments, the anti-EGFR antibody comprises an immunoglobulin heavy chain variable region comprising an amino acid sequence that is at least 70%, 75%, 80%, 85%, 90%, 95%, 96%, 97%, 98%, or 99% identical to the entire variable region and/or the framework region sequence of the amino acid sequence of SEQ ID NO: 15. In certain embodiments, the anti-HGF antibody comprises an immunoglobulin light chain variable region comprising an amino acid sequence that is at least 70%, 75%, 80%, 85%, 90%, 95%, 96%, 97%, 98%, or 99% identical to the entire variable region and/or the framework region sequence of the amino acid sequence of SEQ ID NO: 16.

In each of the foregoing embodiments, it is contemplated herein that immunoglobulin heavy chain variable region sequences and/or light chain variable region sequences that bind EGFR may contain amino acid alterations, e.g., at least 1, 2, 3, 4, 5, or 10 amino acid substitutions, deletions, or additions, e.g., in the framework regions of the heavy and/or light chain variable regions.

In certain embodiments, it is contemplated that a heavy chain variable region sequence, for example, the V_{H} sequence of SEQ ID NO: 15, or any variants thereof, may be covalently linked to a variety of heavy chain constant region sequences known in the art. Similarly, it is contemplated that a light chain variable region sequence, for example, the V_{L} of SEQ ID NO: 16, or any variants thereof, may be covalently linked to a variety of light chain constant region sequences known in the art.

For example, the antibody molecule may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and/or can fix complement. In other embodiments the antibody does not recruit effector cells and/or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the anti-EGFR antibody comprises an immunoglobulin heavy chain comprising the amino acid sequence of SEQ ID NO: 19, or an amino acid sequence that has at least 85%, 90%, 95%, 96%, 97%, 98%, or 99% sequence identity to SEQ ID NO: 19; and/or an immunoglobulin light chain comprising the amino acid sequence of SEQ ID NO: 20, or an amino acid sequence that has at least 85%, 90%, 95%, 96%, 97%, 98%, or 99% sequence identity to SEQ ID NO: 20.

In certain embodiments, the antibody binds human EGFR with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, surface plasmon resonance or bio-layer interferometry. In certain embodiments, the antibody binds human EGFR with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, or from about 0.05 nM to about 0.035 nM, as measured using standard binding assays, for example, surface plasmon resonance or bio-layer interferometry.

In certain embodiments, the invention provides antibodies that bind to the same epitope present in EGFR as that bound by a disclosed antibody. In certain embodiments, the invention provides antibodies that compete for binding to EGFR with a disclosed antibody.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 15, and an immunoglobulin light chain variable region comprising an amino acid sequence that is at least 70%, 75%, 80%, 85%, 90%, 95%, 96%, 97%, 98%, or 99% identical to SEQ ID NO: 16, and (ii) competes for binding to human HGF with and/or binds to same epitope on human EGFR as an antibody comprising an immunoglobulin heavy chain variable region comprising the amino acid sequence of SEQ ID NO: 15, and an immunoglobulin light chain variable region comprising the amino acid sequence of SEQ ID NO: 16.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (i.e., a process referred to as germlining).

In certain embodiments, disclosed antibodies can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

In certain embodiments, the anti-EGFR antibody is cetuximab (ERBITUX®). Cetuximab is an IgG1, chimeric murine-human antibody against EGFR. The amino acid sequence of the CDR_{H1}, CDR_{H2}, and CDR_{H3} sequences of cetuximab are depicted in SEQ ID NO: 9, 10, and 11, respectively. The amino acid sequence of the CDR_{L1}, CDR_{L2}, and CDR_{L3} sequences of cetuximab are depicted in SEQ ID NO: 12, 13, and 14, respectively. The amino acid sequence of the heavy chain variable region of cetuximab is depicted in SEQ ID NO: 15, and the amino acid sequence of the light chain variable region is depicted in SEQ ID NO: 16. The amino acid sequence of the heavy chain of cetuximab is depicted in SEQ ID NO: 19, and the amino acid sequence of the light chain is depicted in SEQ ID NO: 20.

In certain embodiments, the anti-EGFR antibody is imgatuzumab. The amino acid sequence of the heavy chain of imgatuzumab is depicted in SEQ ID NO: 31, and the amino acid sequence of the light chain is depicted in SEQ ID NO: 32.

In certain embodiments, the anti-EGFR antibody is necitumumab. The amino acid sequence of the heavy chain of necitumumab is depicted in SEQ ID NO: 33, and the amino acid sequence of the light chain is depicted in SEQ ID NO: 34.

In certain embodiments, the anti-EGFR antibody is amivantamab (an EGFR and MET bispecific antibody). The amino acid sequences of the heavy chains of amivantamab are depicted in SEQ ID NO: 35 and SEQ ID NO: 36, and the amino acid sequences of the light chains are depicted in SEQ ID NO: 37 and SEQ ID NO: 38.

In certain embodiments, the anti-EGFR antibody is zalutumumab. The amino acid sequence of the heavy chain of zalutumumab is depicted in SEQ ID NO: 39, and the amino acid sequence of the light chain is depicted in SEQ ID NO: 40.

In certain embodiments, the anti-EGFR antibody is panitumumab. The amino acid sequence of the heavy chain of panitumumab is depicted in SEQ ID NO: 41, and the amino acid sequence of the light chain is depicted in SEQ ID NO: 42.

In certain embodiments, the anti-EGFR antibody is nimotuzumab. The amino acid sequence of the heavy chain of nimotuzumab is depicted in SEQ ID NO: 43, and the amino acid sequence of the light chain is depicted in SEQ ID NO: 44.

In certain embodiments, the anti-EGFR antibody is matuzumab. The amino acid sequence of the heavy chain of matuzumab is depicted in SEQ ID NO: 45, and the amino acid sequence of the light chain is depicted in SEQ ID NO: 46.

### IV. Pharmaceutical Compositions

An HGF and/or EGFR inhibitor preferably is combined with a pharmaceutically acceptable carrier.

Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HC1, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

Pharmaceutical compositions containing an HGF and/or EGFR inhibitor disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In one embodiment, the HGF inhibitor, e.g., ficlatuzumab, and the EGFR inhibitor, e.g., cetuximab, are administered by intravenous infusion. The compositions described herein may be administered locally or systemically. Administration will generally be parenteral administration. Preparations for parenteral administration include sterile aqueous or non-aqueous solutions, suspensions, and emulsions. Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

In certain embodiments, an HGF and/or EGFR inhibitor disclosed herein is administered by IV infusion. For IV administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. In certain embodiments, an HGF and/or EGFR inhibitor is lyophilized, and then reconstituted in buffered saline, at the time of administration. Where the composition is lyophilized, filter sterilization can be conducted prior to or following lyophilization and reconstitution.

### V. Methods of Treatment

The invention involves methods of treating cancers with an HGF inhibitor and an EGFR inhibitor. In some embodiments, the invention involves treating recurrent or metastatic HNSCC using ficlatuzumab and cetuximab. In some embodiments, the invention involves treating immune checkpoint inhibitor-resistant recurrent or metastatic HNSCC using ficlatuzumab and cetuximab. In some embodiments, the invention involves treating platinum-resistant recurrent or metastatic HNSCC using ficlatuzumab and cetuximab. In some embodiments, the invention involves treating immune checkpoint inhibitor-resistant and platinum-resistant recurrent or metastatic HNSCC using ficlatuzumab and cetuximab. In some embodiments, an immune checkpoint inhibitor may include a PD-1 inhibitor, a PD-L1 inhibitor, a CTLA-4 inhibitor, a TIM-3 inhibitor, a LAG-3 inhibitor, a TIGIT inhibitor, a VISTA inhibitor, a MR inhibitor, a 2B4 inhibitor, a CD160 inhibitor, a CGEN-15049 inhibitor, a CHK1 inhibitor, a CHK2 inhibitor, a A2aR inhibitor, or any combination thereof. In some embodiments, a PD-1 inhibitor may include nivolumab, pembrolizumab, pidilizumab, REGN2810, PDR001, or any combination thereof. In some embodiments, a PD-L1 inhibitor may include durvalumab, atezolizumab, avelumab, or any combination thereof. In some embodiments, a CTLA-4 inhibitor may include ipilimumab, tremelimumab, AGEN-1884, or any combination thereof. In some embodiments, a TIM-3 inhibitor may include TSR-022, LY3321367, MBG453, or any combination thereof. In some embodiments, a TIGIT inhibitor may include BMS-986207, AGEN17, tiragolumab, MK-7684, OMP-313M32, EOS-448, AB154, or combinations thereof. In some embodiments, a LAG-3 inhibitor may include BMS-986016 REGN3767, IMP321, LAG525, BI754111, favezelimab, or combinations thereof. In some embodiments, a VISTA inhibitor may include CI-8993, HMBD-002, a PSGL-1 antagonist as described in WO 2018/132476, or combinations thereof.

In some embodiments, the invention involves treating recurrent or metastatic HNSCC using ficlatuzumab, cetuximab, and an immune checkpoint inhibitor. In some embodiments, ficlatuzumab and cetuximab are added to a treatment regimen that already includes an immune checkpoint inhibitor to improve the clinical benefit. In some embodiments, an immune checkpoint inhibitor may include a PD-1 inhibitor, a PD-L1 inhibitor, a CTLA-4 inhibitor, a TIM-3 inhibitor, a LAG-3 inhibitor, a TIGIT inhibitor, a VISTA inhibitor, a MR inhibitor, a 2B4 inhibitor, a CD160 inhibitor, a CGEN-15049 inhibitor, a CHK1 inhibitor, a CHK2 inhibitor, a A2aR inhibitor, or any combination thereof. In some embodiments, a PD-1 inhibitor may include nivolumab, pembrolizumab, pidilizumab, REGN2810, PDR001, or any combination thereof. In some embodiments, a PD-L1 inhibitor may include durvalumab, atezolizumab, avelumab, or any combination thereof. In some embodiments, a CTLA-4 inhibitor may include ipilimumab, tremelimumab, AGEN-1884, or any combination thereof. In some embodiments, a TIM-3 inhibitor may include TSR-022, LY3321367, MBG453, or any combination thereof. In some embodiments, a TIGIT inhibitor may include BMS-986207, AGEN17, tiragolumab, MK-7684, OMP-313M32, EOS-448, AB154, or combinations thereof. In some embodiments, a LAG-3 inhibitor may include BMS-986016, REGN3767, IMP321, LAG525, BI754111, favezelimab, or combinations thereof. In some embodiments, a VISTA inhibitor may include CI-8993, HMBD-002, a PSGL-1 antagonist as described in WO 2018/132476, or combinations thereof. In some embodiments, the platinum is arboplatin, oxaliplatin, cisplatin, nedaplatin, triplatin tetranitrate, lobaplatin, phenanthriplatin, picoplatin, and satraplatin.

Methods and compositions of the invention can be used to treat any type of cancer, including, but not limited to, lung cancer, liver cancer, ovarian cancer, prostate cancer, testicular cancer, gallbladder cancer, sarcoma, Ewing sarcoma, thyroid cancer, melanoma, skin cancer, pancreatic cancer; gastrointestinal/stomach (GIST) cancer, lymphoma, head and neck cancer, glioma or brain cancer, colon cancer, rectal cancer, colorectal cancer, breast cancer, renal cell carcinoma or kidney cancer. In one embodiment, the cancer is head and neck squamous cell carcinoma (HNSCC). In some embodiments, the HNSCC is hypopharyngeal cancer, laryngeal cancer, lip and oral cavity cancer, nasopharyngeal cancer, oropharyngeal cancer, paranasal sinus and/or nasal cavity cancer, or salivary gland cancer. In some embodiments, the cancer is colon cancer, rectal cancer, and/or colorectal cancer. In some embodiments, the cancer is an HPV negative cancer. In some embodiments, the cancer is colon cancer, rectal cancer, and/or colorectal cancer which is treated with cetuximab and ficlatuzumab according to the methods disclosed herein. In some embodiments, the cancer is an HPV negative cancer which is treated with cetuximab and ficlatuzumab according to the inventions disclosed herein. In some embodiments, the cancer is an HPV negative rectal, colon, or colorectal cancer which is treated with cetuximab and ficlatuzumab according to the inventions disclosed herein.

In certain embodiments, a cancer, tumor, disease, or subject treated with a method or composition of the invention is resistant to one or more immune checkpoint inhibition therapies. In some embodiments, immune checkpoint inhibitor resistance refers to a subject with HNSCC who does not respond to immune checkpoint inhibitor treatment or who responded and then stopped responding. In certain embodiments, immune checkpoint inhibitor resistance refers to: (i) disease persistence or recurrence within 6 months of completing definitive radiotherapy for locally advanced disease, for example, an HNSCC, where radiation included concurrent immune checkpoint inhibitor therapy, or (ii) disease progression during, or within 6 months, of immune checkpoint inhibitor treatment in a recurrent/metastatic setting, for example, a recurrent/metastatic HNSCC. In some embodiments, prior immune checkpoint inhibitor therapy exposure may have occurred in any line of therapy (first line, second line, etc.) and immune checkpoint inhibitor therapy is not required to be the most recent therapy received in order for a cancer or tumor to be classified as immune checkpoint inhibitor therapy resistant. In certain embodiments, a cancer, tumor, disease, e.g., HNSCC or subject, for example, a subject with HNSCC, treated with a method or composition of the invention is immune checkpoint inhibitor therapy-ineligible, i.e., not an acceptable candidate for treatment with an immune checkpoint inhibitor therapy, e.g., due to medical comorbidities. In some embodiments, an immune checkpoint inhibitor may include a PD-1 inhibitor, a PD-L1 inhibitor, a CTLA-4 inhibitor, a TIM-3 inhibitor, a LAG-3 inhibitor, a TIGIT inhibitor, a VISTA inhibitor, a MR inhibitor, a 2B4 inhibitor, a CD160 inhibitor, a CGEN-15049 inhibitor, a CHK1 inhibitor, a CHK2 inhibitor, a A2aR inhibitor, or any combination thereof. In some embodiments, a PD-1 inhibitor may include nivolumab, pembrolizumab, pidilizumab, REGN2810, PDR001, or any combination thereof. In some embodiments, a PD-L1 inhibitor may include durvalumab, atezolizumab, avelumab, or any combination thereof. In some embodiments, a CTLA-4 inhibitor may include ipilimumab, tremelimumab, AGEN-1884, or any combination thereof. In some embodiments, a TIM-3 inhibitor may include TSR-022, LY3321367, MBG453, or any combination thereof. In some embodiments, a TIGIT inhibitor may include BMS-986207, AGEN17, tiragolumab, MK-7684, OMP-313M32, EOS-448, AB154, or combinations thereof. In some embodiments, a LAG-3 inhibitor may include BMS-986016, REGN3767, IMP321, LAG525, BI754111, favezelimab, or combinations thereof. In some embodiments, a VISTA inhibitor may include CI-8993, HMBD-002, a PSGL-1 antagonist as described in WO 2018/132476, or combinations thereof.

In certain embodiments, a cancer, tumor, disease, or subject treated with a method or composition of the invention is platinum-resistant. In certain embodiments, platinum resistance refers to: (i) disease persistence or recurrence within 6 months of completing definitive radiotherapy for locally advanced disease, for example, an HNSCC, where platinum chemotherapy was administered as a component of induction and/or concurrent systemic treatment, or (ii) disease progression during, or within 6 months, of treatment with platinum chemotherapy in a recurrent/metastatic setting, for example, a recurrent/metastatic HNSCC. In some embodiments, prior platinum exposure may occur in any line of therapy (first line, second line, etc.) and is not required to be the most recent therapy received in order for a cancer or tumor to be classified as platinum-resistant. In certain embodiments, a cancer, tumor, disease, e.g., HNSCC or subject, for example, a subject with HNSCC, treated with a method or composition of the invention is platinum-ineligible, i.e., not an acceptable candidate for platinum chemotherapy, e.g., due to medical comorbidities. Exemplary platinum chemotherapies include carboplatin, oxaliplatin, cisplatin, nedaplatin, triplatin tetranitrate, lobaplatin, phenanthriplatin, picoplatin, and satraplatin.

In certain embodiments, a cancer, tumor, disease, or subject treated with a method or composition of the invention is cetuximab-resistant. In some embodiments, cetuximab-resistant refers to a subject with HNSCC who does not respond to cetuximab treatment or who responded and then stopped responding. In certain embodiments, cetuximab resistance refers to: (i) disease persistence or recurrence within 6 months of completing definitive radiotherapy for locally advanced disease, for example, an HNSCC, where radiation included concurrent cetuximab, or (ii) disease progression during, or within 6 months, of cetuximab treatment in a recurrent/metastatic setting, for example, a recurrent/metastatic HNSCC. In some embodiments, prior cetuximab exposure may occur in any line of therapy (definitive-intent or curative-intent treatment, or first line, second line, etc.) and cetuximab is not required to be the most recent therapy received in order for a cancer or tumor to be classified as cetuximab-resistant. In certain embodiments, a cancer, tumor, disease, e.g., HNSCC or subject, for example, a subject with HNSCC, treated with a method or composition of the invention is cetuximab-ineligible, i.e., not an acceptable candidate for treatment with cetuximab, e.g., due to medical comorbidities. In certain embodiments, a cetuximab-resistant HNSCC, e.g., recurrent/metastatic HNSCC, is treated with cetuximab in combination with ficlatuzumab according to the methods of the invention.

In certain embodiments, a cancer, tumor, disease, or subject treated with a method or composition of the invention is human papillomavirus (HPV)-negative. In certain embodiments, HPV positive refers to a cancer, tumor, or disease, e.g., an HNSCC, that is p16 positive, as measured by immunohistochemistry, meaning that ≥70% of cancer, tumor, or disease cells demonstrate diffuse nuclear and cytoplasmic staining with a p16 antibody. In certain embodiments, HPV negative refers to a cancer, tumor, or disease, e.g., an HNSCC, that is not p16 positive, as measured by immunohistochemistry, meaning that <70% of cancer, tumor, or disease cells demonstrate diffuse nuclear and cytoplasmic staining with a p16 antibody. In certain embodiments, HPV positive refers to an HNSCC, that (i) has an oropharynx or unknown primary site, and (ii) is p16 positive, as measured by immunohistochemistry, meaning that ≥70% of cancer, tumor, or disease cells demonstrate diffuse nuclear and cytoplasmic staining with a p16 antibody. In certain embodiments, HPV negative refers to an HNSCC that (i) is an oropharynx or unknown primary site and is not p16 positive, as measured by immunohistochemistry, meaning that <70% of cancer, tumor, or disease cells demonstrate diffuse nuclear and cytoplasmic staining with a p16 antibody. In certain embodiments, HPV negative refers to an HNSCC that is not of an oropharynx or unknown primary site (i.e., an HNSCC of a known primary site other than the oropharynx is presumed to be HPV negative). For example, primary site oral, laryngeal, and hypopharyngeal HNSCC is presumed to be HPV-negative. In some embodiments, an HNSCC is HPV-negative if it is not p16 positive as measured by immunohistochemistry, meaning that <70% of cancer, tumor, or disease cells demonstrate diffuse nuclear and cytoplasmic staining with a p16 antibody. In certain embodiments, the HPV status of the cancer, tumor, disease, or subject is assessed prior to treatment with a method or composition of the invention. Any known immunohistochemistry methods may be used to identify the HPV status of a subject according to the present disclosure. For example, p16 immunohistochemical staining with a p16 antibody can be used to assess HPV status, or analysis of tumor DNA can be used to determine HPV status. Methods for determining the HPV status of HNSCC are known in the art and can be used to determine HPV status according to the invention. See, e.g., Jordan et al. Am J Surg Pathol. (2012) 36 (7): 945-954.

In certain embodiments, a cancer, tumor, disease, or subject treated with a method or composition of the invention is human papillomavirus (HPV)-negative. In certain embodiments, HPV positive refers to a cancer, tumor, or disease where the presence of HPV is detected by the presence of nucleic acids (e.g., DNA or RNA) of HPV in the subject's tumor, e.g., in an HNSCC in a subject. In certain embodiments, HPV negative refers to a cancer, tumor, or disease where HPV nucleic acids (e.g., DNA or RNA) are not detected in the tumor, e.g., in an HNSCC of the subject. In certain embodiments, HPV negative HNSCC refers to an HNSCC that (i) does not have an oropharynx or unknown primary site (i.e., an HNSCC of a known primary site other than the oropharynx is presumed to be HPV negative), and/or (ii) where HPV nucleic acids (e.g., DNA or RNA) are not detected in the tumor, e.g., in an HNSCC of the subject. In certain embodiments, the HPV status of the cancer, tumor, disease, or subject is assessed prior to treatment with a method or composition of the invention. Any known methods of assessing the presence, or absence, of HPV nucleic acids (e.g., DNA, RNA, etc.) in a subject may be used to identify the HPV status of a subject according to the present disclosure (e.g., PCR, in situ hybridization, etc.). See, e.g., Jordan et al. Am J Surg Pathol. (2012) 36 (7): 945-954.

In one embodiment, subjects (e.g., HPV negative subjects) treated with an HGF inhibitor and an EGFR inhibitor according to the methods of the invention, e.g., ficlatuzumab and cetuximab, have an overall response rate (ORR) of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In certain embodiments, the overall response rate (ORR) is at least 35%.

In one embodiment, subjects (e.g., HPV negative subjects) treated with an HGF inhibitor and an EGFR inhibitor according to the methods of the invention, e.g., ficlatuzumab and cetuximab, have a median overall survival (OS) rate of at least one month, at least two months, at least three months, at least four months, at least 5 months, at least 6 months, at least 7 months, at least eight months, at least nine months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, or at least 60 months.

In certain embodiments, subjects (e.g., HPV negative subjects) treated with an HGF inhibitor and an EGFR inhibitor according to the methods of the invention, e.g., ficlatuzumab and cetuximab, have a median overall survival (OS) rate of from about 60 months to about one month, from about 60 months to about two months, from about 60 months to about three months, from about 60 months to about four months, from about 60 months to about 5 months, from about 60 months to about 6 months, from about 60 months to about 7 months, from about 60 months to about 8 months, from about 60 months to about 9 months, from about 60 months to about 10 months, from about 60 months to about 20 months, from about 30 months to about one month, from about 30 months to about two months, from about 30 months to about three months, from about 30 months to about four months, from about 30 months to about 5 months, from about 30 months to about 6 months, from about 30 months to about 7 months, from about 30 months to about 8 months, from about 30 months to about 9 months, from about 30 months to about 10 months, from about 30 months to about 20 months, from about 15 months to about one month, from about 15 months to about two months, from about 15 months to about three months, from about 15 months to about four months, from about 15 months to about 5 months, from about 15 months to about 6 months, from about 15 months to about 7 months, from about 15 months to about 8 months, from about 15 months to about 9 months, from about 15 months to about 10 months, from about 10 months to about one month, from about 10 months to about two months, from about 10 months to about three months, from about 10 months to about four months, from about 10 months to about 5 months, from about 10 months to about 6 months, from about 10 months to about 7 months, from about 10 months to about 8 months, from about 10 months to about 9 months, from about 6 months to about one month, from about 6 months to about two months, from about 6 months to about three months, from about 6 months to about four months, or from about 6 months to about 5 months.

In one embodiment, subjects (e.g., HPV negative subjects) treated with an HGF inhibitor and an EGFR inhibitor according to the methods of the invention, e.g., ficlatuzumab and cetuximab, have a median progression-free survival (PFS) of at least one month, at least two months, at least three months, at least four months, at least 5 months, at least 6 months, at least 7 months, at least eight months, at least nine months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, or at least 60 months.

In certain embodiments, subjects (e.g., HPV negative subjects) treated with an HGF inhibitor and an EGFR inhibitor according to the methods of the invention, e.g., ficlatuzumab and cetuximab, have a median progression-free survival (PFS) of from about 60 months to about one month, from about 60 months to about two months, from about 60 months to about three months, from about 60 months to about four months, from about 60 months to about 5 months, from about 60 months to about 6 months, from about 60 months to about 7 months, from about 60 months to about 8 months, from about 60 months to about 9 months, from about 60 months to about 10 months, from about 60 months to about 20 months, from about 30 months to about one month, from about 30 months to about two months, from about 30 months to about three months, from about 30 months to about four months, from about 30 months to about 5 months, from about 30 months to about 6 months, from about 30 months to about 7 months, from about 30 months to about 8 months, from about 30 months to about 9 months, from about 30 months to about 10 months, from about 30 months to about 20 months, from about 15 months to about one month, from about 15 months to about two months, from about 15 months to about three months, from about 15 months to about four months, from about 15 months to about 5 months, from about 15 months to about 6 months, from about 15 months to about 7 months, from about 15 months to about 8 months, from about 15 months to about 9 months, from about 15 months to about 10 months, from about 10 months to about one month, from about 10 months to about two months, from about 10 months to about three months, from about 10 months to about four months, from about 10 months to about 5 months, from about 10 months to about 6 months, from about 10 months to about 7 months, from about 10 months to about 8 months, from about 10 months to about 9 months, from about 6 months to about one month, from about 6 months to about two months, from about 6 months to about three months, from about 6 months to about four months, or from about 6 months to about 5 months. In one embodiment, the median progression-free survival (PFS) is at least 4 months.

According to certain embodiments of the invention, treatment with an HGF inhibitor and an EGFR inhibitor is indicated as long as a clinical benefit is observed in the subject or until unacceptable toxicity occurs.

Exemplary effective amounts, or dosages of an HGF inhibitor (e.g., anti-HGF antibody or antigen binding fragment thereof, e.g., ficlatuzumab) include about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, or about 25 mg/kg. In certain embodiments, exemplary effective amounts, or dosages of an HGF inhibitor (e.g., anti-HGF antibody or antigen binding fragment thereof, e.g., ficlatuzumab) include from about 25 mg/kg to about 0.5 mg/kg, from about 25 mg/kg to about 1 mg/kg, from about 25 mg/kg to about 2 mg/kg, from about 25 mg/kg to about 3 mg/kg, from about 25 mg/kg to about 4 mg/kg, from about 25 mg/kg to about 5 mg/kg, from about 25 mg/kg to about 10 mg/kg, from about 25 mg/kg to about 15 mg/kg, from about 25 mg/kg to about 20 mg/kg, from about 20 mg/kg to about 0.5 mg/kg, from about 20 mg/kg to about 1 mg/kg, from about 20 mg/kg to about 2 mg/kg, from about 20 mg/kg to about 3 mg/kg, from about 20 mg/kg to about 4 mg/kg, from about 20 mg/kg to about 5 mg/kg, from about 20 mg/kg to about 10 mg/kg, from about 20 mg/kg to about 15 mg/kg, from about 15 mg/kg to about 0.5 mg/kg, from about 15 mg/kg to about 1 mg/kg, from about 15 mg/kg to about 2 mg/kg, from about 15 mg/kg to about 3 mg/kg, from about 15 mg/kg to about 4 mg/kg, from about 15 mg/kg to about 5 mg/kg, or from about 15 mg/kg to about 10 mg/kg. In some embodiments, an exemplary effective amount, or dosage of an HGF inhibitor (e.g., anti-HGF antibody or antigen binding fragment thereof, e.g., ficlatuzumab) is about 20 mg/kg. In some embodiments, an exemplary effective amount, or dosage of an HGF inhibitor (e.g., anti-HGF antibody or antigen binding fragment thereof, e.g., ficlatuzumab) is about 10 mg/kg. In some embodiments, an exemplary effective amount, or dosage of an HGF inhibitor (e.g., anti-HGF antibody or antigen binding fragment thereof, e.g., ficlatuzumab) is about 15 mg/kg.

In some embodiments, the dosage of an ficlatuzumab is about 20 mg/kg and the dosage of cetuximab is 500 mg/m². In some embodiments, the dosage of an ficlatuzumab is about 15 mg/kg and the dosage of cetuximab is 500 mg/m². In some embodiments, the dosage of an ficlatuzumab is about 10 mg/kg and the dosage of cetuximab is 500 mg/m². In some embodiments, the dosage of an ficlatuzumab is about 20 mg/kg and the dosage of cetuximab is 400 mg/m². In some embodiments, the dosage of an ficlatuzumab is about 15 mg/kg and the dosage of cetuximab is 400 mg/m². In some embodiments, the dosage of an ficlatuzumab is about 10 mg/kg and the dosage of cetuximab is 400 mg/m². In some embodiments, the dosage of an ficlatuzumab is about 20 mg/kg and the dosage of cetuximab is 300 mg/m². In some embodiments, the dosage of an ficlatuzumab is about 15 mg/kg and the dosage of cetuximab is 300 mg/m². In some embodiments, the dosage of an ficlatuzumab is about 10 mg/kg and the dosage of cetuximab is 300 mg/m². In some embodiments, the aforementioned dosages of ficlatuzumab and cetuximab are provided every two weeks. In some embodiments, the aforementioned dosages of ficlatuzumab and cetuximab are provided every two weeks on the same day. In some embodiments, the aforementioned dosages of ficlatuzumab and cetuximab are provided every two weeks simultaneously or sequentially on the same day.

Exemplary treatment regimens for an HGF inhibitor (e.g., anti-HGF antibody or antigen binding fragment thereof, e.g., ficlatuzumab) include administration of an effective dosage about every week, about every two weeks, about every three weeks, about every four weeks, about every 5 weeks, about every 6 weeks, about every 7 weeks, or about every 8 weeks. In some embodiments, exemplary treatment regimens for an HGF inhibitor (e.g., anti-HGF antibody or antigen binding fragment thereof, e.g., ficlatuzumab) include administration about every one to two weeks, about every two to three weeks, or about every three to four weeks. In some embodiments, an exemplary treatment regimen for an HGF inhibitor (e.g., anti-HGF antibody or antigen binding fragment thereof, e.g., ficlatuzumab) includes administration at about every two weeks. In some embodiments, an exemplary treatment regimen for an HGF inhibitor (e.g., anti-HGF antibody or antigen binding fragment thereof, e.g., ficlatuzumab) includes administration every two weeks.

Exemplary effective amounts, or dosages of an EGFR inhibitor (e.g., anti-EGFR antibody or antigen binding fragment thereof, e.g., cetuximab) include about 200 mg/m², about 250 mg/m², about 300 mg/m², about 400 mg/m², about 500 mg/m², about 600 mg/m², about 700 mg/m², or about 800 mg/m². In certain embodiments, exemplary effective amounts, or dosages of an EGFR inhibitor (e.g., anti-EGFR antibody or antigen binding fragment thereof, e.g., cetuximab) include from about 800 mg/m² to about 200 mg/m² from about 800 mg/m² to about 300 mg/m², from about 800 mg/m² to about 400 mg/m², from about 800 mg/m² to about 500 mg/m², from about 800 mg/m² to about 600 mg/m², from about 800 mg/m² to about 700 mg/m², from about 600 mg/m² to about 200 mg/m², from about 600 mg/m² to about 300 mg/m², from about 600 mg/m² to about 400 mg/m², from about 700 mg/m² to about 400 mg/m², from about 700 mg/m² to about 250 mg/m², from about 600 mg/m² to about 500 mg/m², from about 500 mg/m² to about 300 mg/m², or from about 300 mg/m² to about 200 mg/m². In certain embodiments, an exemplary effective amount, or dosage of an EGFR inhibitor (e.g., anti-EGFR antibody or antigen binding fragment thereof, e.g., cetuximab) is about 500 mg/m².

Exemplary treatment regimens for an EGFR inhibitor (e.g., anti-EGFR antibody or antigen binding fragment thereof, e.g., cetuximab) include administration of an effective dosage about every week, about every two weeks, about every three weeks, about every four weeks, about every 5 weeks, about every 6 weeks, about every 7 weeks, or about every 8 weeks. In some embodiments, exemplary treatment regimens for an EGFR inhibitor (e.g., anti-EGFR antibody or antigen binding fragment thereof, e.g., cetuximab) include administration about every one to two weeks, about every two to three weeks, or about every three to four weeks. In some embodiments, an exemplary treatment regimen for an EGFR inhibitor (e.g., anti-EGFR antibody or antigen binding fragment thereof, e.g., cetuximab) is about every two weeks. In one embodiment, the EGFR inhibitor, e.g. cetuximab is administered on the same day as the HGF inhibitor, e.g., ficlatuzumab. For example, the EGFR inhibitor, e.g. cetuximab is administered concurrently with the HGF inhibitor, e.g., ficlatuzumab. In one embodiment, cetuximab and ficlatuzumab are administered on the same day every two weeks. In one embodiment, cetuximab and ficlatuzumab are administered on the same day every three weeks. In one embodiment, cetuximab and ficlatuzumab are administered on the same day every four weeks.

The amount administered will depend on variables such as the type and extent of disease or indication to be treated, the overall health of the patient, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study. Dosing frequency can vary, depending on factors such as route of administration, dosage amount, and the disease being treated. Exemplary dosing frequencies are once per day, once every other day, once every three days, once every four days, once every five days, once every six days, once per week and once every two weeks.

The HGF inhibitor, e.g., ficlatuzumab and the anti-EGFR antibody, e.g., cetuximab may, for example, be administered concurrently for at least one cycle of treatment. In one embodiment, a treatment cycle is 4 weeks which includes administrations of cetuximab and ficlatuzumab at 2 weeks and 4 weeks. The HGF inhibitor (e.g., anti-HGF antibody such as ficlatuzumab) or EGFR inhibitor (e.g., anti-EGFR antibody such as cetuximab) may, for example, be administered sequentially for at least one cycle of treatment. In certain embodiments, the HGF inhibitor (e.g., anti-HGF antibody, e.g., ficlatuzumab) is administered after the EGFR inhibitor (e.g., anti-EGFR antibody, e.g., cetuximab) for at least one cycle of treatment, for example, the HGF inhibitor (e.g., ficlatuzumab) is administered at least 15 minutes, at least 30 minutes, at least 60 minutes, at least 120 minutes, at least 180 minutes, at least 240 minutes, or at least 300 minutes, or from about 30 to about 60 minutes, after completion of the administration of the EGFR inhibitor (e.g., cetuximab).

As discussed herein, in certain embodiments, a subject may receive a first line therapy which is determined to be ineffective (e.g., due to primary resistance, acquired resistance, etc.) prior to receiving treatment with the disclosed compositions and/or methods. A first line therapy is a treatment generally accepted in the medical field for initial treatment of a given disease or disorder (e.g., cancer). As used herein, with regard to recurrent/metastatic HNSCC, a first line therapy refers to an initial treatment for recurrent/metastatic HNSCC (i.e., an initial palliative treatment), and does not take into account any prior treatments for non-recurrent or localized HNSCC (i.e. any prior definitive-intent or curative-intent treatments). Similarly a second line therapy for recurrent/metastatic HNSCC, refers to a second treatment for recurrent/metastatic HNSCC (i.e., a second palliative treatment), and does not take into account any prior treatments for non-recurrent or localized HNSCC (i.e., any prior definitive-intent or curative-intent treatments). In some embodiments, a first line therapy includes antibody (e.g., monoclonal antibody) or antibody fragment therapy, immune-checkpoint therapy, cancer vaccines, adoptive cell transfer, cytokine therapy, or any combinations thereof. As described herein, in some embodiments, a first line therapy includes an immune checkpoint inhibitor therapy and/or a platinum therapy.

According to one embodiment, a subject is treated according to the methods of the invention as long as the subject experiences a clinical benefit or until the subject experiences unacceptable toxicity. For example, to treat recurrent/metastatic HNSCC the subject is treated with cetuximab and ficlatuzumab every two weeks as long as the subject experiences a clinical benefit or until the subject experiences unacceptable toxicity. For example, to treat recurrent/metastatic HNSCC the subject is treated with a cetuximab and ficlatuzumab treatment cycle (i.e., which is four weeks with cetuximab and ficlatuzumab being administered every 2 weeks, i.e., twice in the treatment cycle), and receives 1, 2, 3, 4 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more treatment cycles. For example, to treat recurrent/metastatic HNSCC the subject receives treatment cycles of cetuximab and ficlatuzumab until the HNSCC progresses.

### EXAMPLES

The present disclosure will be further illustrated in the following Examples which are given for illustration purposes only and are not intended to limit the disclosure in any way.

### Example 1—Study Examining the Efficacy of Ficlatuzumab and Cetuximab in the Treatment of Pan-Refractory, Advanced HNSCC

Provided herein is an exemplary protocol and exemplary results for treating subjects in need with combination anti-HGF antibody and anti-EGFR antibody therapy.

A phase Ib trial showed safety and preliminary efficacy of cetuximab and ficlatuzumab in cetuximab-resistant, advanced HNSCC. (see Bauman et al., Journal of Clinical Oncology 35, no. 15 suppl (May 20, 2017) 6038-6038, and Bauman et al., Cancers (Basel). 2020:12(6):1537. Published 2020 Jun. 11. doi:10.3390/cancers12061537). The promising clinical activity observed in patients with cetuximab-resistant, recurrent/metastatic HNSCC during phase Ib led to a randomized, phase II study evaluating the efficacy of ficlatuzumab, with or without cetuximab, in patients with cetuximab-resistant, recurrent/metastatic HNSCC. The recommended phase II dose was 20 mg/kg for ficlatuzumab and 500 mg/m² for cetuximab every two weeks (“the combination arm”) with a control arm receiving only 20 mg/kg ficlatuzumab every two weeks Overall response rate (ORR) and median progression-free survival (mPFS) were 17% and 5.4 months. An increase in peripheral T cells, particularly the CD8+ subset was associated with treatment response whereas expansion of a distinct myeloid population was associated with progression.

### 1. Study Design

a. Study Objectives

The primary study objective was to assess the efficacy of ficlatuzumab, with or without concurrent cetuximab, in patients with cetuximab-resistant, recurrent/metastatic HNSCC as measured by Progression-Free Survival (PFS).

Secondary study objectives included description of toxicity and patient-reported quality of life and evaluation of response rate and overall survival in both treatment arms.

b. Inclusion/Exclusion Criteria

Each patient enrolled in the study met all of the following inclusion criteria:
Patients must have had a histologically confirmed HNSCC from any primary site. Basaloid, poorly differentiated, and undifferentiated carcinoma histologies were accepted. Nasopharyngeal carcinoma, WHO Type I and II (keratinizing, non-EBV positive), will be included. Paranasal sinus, lip and external auditory canal sites were included. Squamous cell carcinoma of unknown primary, clearly related to the head and neck, were included.
Recurrent/metastatic disease, fulfilling at least one of the criteria defined below:
   Incurable disease as assessed by surgical or radiation oncology
   Metastatic (M1) disease
   Persistent or progressive disease following curative-intent radiation, and not a candidate for surgical salvage due to incurability or morbidity. Patients who declined radical surgery were eligible.
For patients with oropharyngeal primary site or unknown primary site only: tumoral

HPV status must have been known, as established by the local site. Acceptable standards included p16 immunohistochemistry (where a tumor was classified as p16-positive when showing diffuse nuclear and cytoplasmic staining in at least 70% of tumor cells) and/or assessment of HPV DNA.
Patients had to be cetuximab-resistant by fulfilling at least one of the criteria defined below:
   Disease persistence or recurrence within 6 months of completing definitive radiotherapy for locally advanced disease. Radiation must have included concurrent cetuximab. Induction chemotherapy, if given, may or may not have included cetuximab.
   Disease progression during, or within 6 months, of cetuximab treatment in the recurrent/metastatic setting.
   Prior cetuximab exposure may have occurred in any line of therapy (first line, second line, etc.) and cetuximab was not required to be the most recent therapy received.
Patients had to be platinum-resistant or platinum-ineligible by fulfilling at least one of the criteria defined below:
   Disease persistence or recurrence within 6 months of completing definitive radiotherapy for locally advanced disease, where platinum chemotherapy was administered as a component of induction and/or concurrent systemic treatment.
   Disease progression during, or within 6 months, of treatment with platinum chemotherapy (e.g., carboplatin or cisplatin) in the recurrent/metastatic setting.
   The patient was not an acceptable candidate for platinum chemotherapy due to medical comorbidities, in the judgment of the local investigator.
   Prior platinum exposure may have occurred in any line of therapy (first line, second line, etc.) and was not required to be the most recent therapy received.
Prior exposure to immunotherapy, including anti-PD1/PDL1, anti-CTLA4, anti-TNFR antibodies or other investigational immunotherapies, was acceptable.
Eastern Cooperative Oncology Group Performance Status 0-1 at time of informed consent.
Age ≥18 years.
Patients must have consented to a research biopsy of tumor tissue at baseline, for conduct of correlative studies. Archived biopsy material was only substituted if no interval anti-cancer systemic therapy had been administered.
Measurable disease per RECIST criteria, version 1.1 (see, e.g., section 6 of Eisenhauer, E. A. et al. Eur J Cancer (2009) 45:228-247).
Patients must have had the following laboratory values measured within 28 days of registration:
   Absolute neutrophil count (ANC)≥1500/mm³
   Platelet count (PLT)≥75,000/mm³
   Creatinine clearance≥40 ml/min as determined by 24-hour collection or estimated by the Cockraft-Gault formula:

$$Calculated Creatinine Clearance = [ ( 140 - age ) \times ( actual body weight in kg ) \times ( 0.85 if female ) ] / ( 72 \times serum creatinine )$$

Serum bilirubin≤1.5 times upper-limit of normal (ULN)
AST (aspartate aminotransferase) and ALT (alanine aminotransferase) ≤3 times ULN
No prior severe infusion reaction to cetuximab or a monoclonal antibody
Written informed consent was obtained from all patients prior to beginning therapy. Patients must have had the ability to understand and the willingness to sign a written informed consent document.
If a woman of childbearing potential, documentation of negative pregnancy within 14 days prior to registration was required. A negative pregnancy test was confirmed within 3 days of the first dose of ficlatuzumab. Sexually active women of childbearing potential agreed to use adequate contraceptive measures, while on study and for 30 days after the last dose of study drug.

Patients meeting any of the following exclusion criteria were not enrolled in the study:
Nasopharyngeal primary site, if WHO Type III (non-keratinizing and EBV-positive as established at the local site)
History of severe allergic or anaphylactic reactions or hypersensitivity to recombinant proteins or excipients in the investigational agent.
Prior treatment with an HGF/cMet inhibitor such as rilotumumab, crizotinib, MetMAb, or ARQ197.
Uncontrolled central nervous system (CNS) metastases, including leptomeningeal metastases, are not allowed. Subjects with previously treated brain metastases were allowed if the brain metastases were stable without steroid treatment for at least 2 weeks (radiotherapy or surgery).
Failure to recover to Grade 1 or baseline from all toxic effects of previous chemotherapy, radiation therapy, biologic therapy, immunotherapy, and/or experimental therapy, with the exception of: alopecia, Grade≤2 peripheral neuropathy, Grade≤2 cetuximab-related rash or other skin changes, hypomagnesemia (acceptable values detailed below), hypokalemia (acceptable values detailed below), and the acceptable hematologic values summarized above. A washout period of 2 weeks from prior cetuximab was required; a washout period of 3 weeks from any prior cytotoxic chemotherapy, targeted therapy, immunotherapy or investigational drug was required.
Significant pulmonary disease, including pulmonary hypertension or interstitial pneumonitis.
Decreased serum albumin <30 g/L (<3 g/dL)
Peripheral edema ≥Grade 2 per NCI-CTCAE version 4.0.
Significant electrolyte imbalance prior to enrollment (note that patients may be supplemented to achieve acceptable electrolyte values):
   Hypomagnesemia <1.2 mg/dL or 0.5 mmol/L.
   Hypocalcemia <8.0 mg/dL or 2.0 mmol/L.
   Hypokalemia <3.0 mmol/L.
Significant cardiovascular disease, including:
   Cardiac failure New York Heart Association (NYHA) class III or IV.
   Myocardial infarction, severe or unstable angina within 6 months prior to Study Day 1.
   History of serious ventricular arrhythmia (i.e., ventricular tachycardia or ventricular fibrillation).
   Cardiac arrhythmia requiring anti-arrhythmic medication(s). Note that beta-blockers, calcium channel blockers, and digoxin administered for the purpose rate control of supraventricular tachycardia, including atrial fibrillation and atrial flutter, are not classified as anti-arrhythmic medications for purposes of trial eligibility.
Significant thrombotic or embolic events within 4 weeks prior to Study Day 1. Significant thrombotic or embolic events included but were not limited to stroke or transient ischemic attack (TIA). Catheter-related thrombosis was not a cause for exclusion. Diagnosis of deep vein thrombosis or pulmonary embolism was allowed if it occurred >4 weeks prior to Study Day 1 and the patient was asymptomatic and stable on anti-coagulation therapy.
Any other medical condition (e.g., alcohol abuse) or psychiatric condition that, in the opinion of the Investigator, would have interfered with the subject's participation in the trial or interfered with the interpretation of trial results.
History of second malignancy within 2 years prior to Study Day 1 (except for excised and cured non-melanoma skin cancer, carcinoma in situ of breast or cervix, superficial bladder cancer, Stage I differentiated thyroid cancer that is resected or observed, or pT1a/pT1b prostate cancer comprising <5% of resected tissue with normal prostate specific antigen (PSA) since resection, or cT1a/cT1b prostate cancer treated with brachytherapy or external beam radiation therapy with normal PSA since radiation).
Major surgery within 6 weeks prior to Study Day 1 (subjects must have completely recovered from any previous surgery prior to Study Day 1).
Active infection requiring systemic antibiotics or antifungals within 7 days prior to first dose of study drug. Exception: tetracycline family antibiotics (tetracycline, doxycycline, minocycline) administered for the management of cetuximab-related rash could be continued per the Investigator's judgment.
HIV-positive patients receiving combination anti-retroviral therapy were excluded from the study because of possible drug interactions with study drugs.
Women could not be pregnant or breastfeeding because ficlatuzumab and/or cetuximab could be harmful to the fetus or the nursing infant. Pregnant women were excluded from this study because ficlatuzumab and/or cetuximab had the potential for teratogenic or abortifacient effects.
   c. Randomization

Randomization to ficlatuzumab vs. the combination of ficlatuzumab and cetuximab occurred at the UACC Biostatistics Shared Resource. Patients were stratified by HPV status, a known prognostic factor in recurrent/metastatic HNSCC.

For purposes of stratification, HPV-positive HNSCC was assessed by p16 status performed per standard of care at the local site. To be classified as HPV-positive, patients had to meet BOTH of the following criteria: 1) either oropharynx or unknown primary site; AND 2) p16+ by immunohistochemistry, where ≥70% of tumor cells demonstrate diffuse nuclear and cytoplasmic staining with p16 antibody. For purposes of stratification, the remainder of patients were classified as HPV-negative.

d. Treatment Plan

Ficlatuzumab was administered as an IV infusion. Ficlatuzumab was administered at the dose of 20 mg/kg IV every 2 weeks (+/−3 days), beginning on the same day as the first dose of cetuximab. Ficlatuzumab was administered over 30-60 minutes. Protocol-specified dose modifications were permitted. See Table 1 below for dose reduction levels. Cetuximab will be administered first. Ficlatuzumab will be administered 30-60 minutes after the completion of the cetuximab infusion.

Cetuximab was administered as an IV infusion. Cetuximab was administered at the dose of 500 mg/m² IV every 2 weeks (+/−3 days), beginning on the same day as the first dose of ficlatuzumab. The first dose was administered over 120 minutes (+/−15 minutes) and subsequent doses were permitted to be infused over 60 minutes (+/−15 minutes). Protocol-specified dose modifications were permitted. See Table 1 below for dose reduction levels. Cetuximab was administered at the dose of 500 mg/m² IV every 2 weeks (+/−3 days), beginning on the same day as the first dose of ficlatuzumab. The first dose was administered over 120 minutes (+/−15 minutes) and subsequent doses were permitted to be infused over 60 minutes (+/−15 minutes). Protocol-specified dose modifications were permitted. See Table 1 below for dose reduction levels.

In the absence of treatment delays due to adverse event(s), treatment continued until disease progression or until one of the following criteria applies:
Intercurrent illness that prevented further administration of treatment,
Unacceptable adverse event(s),
Patient elected to withdraw from the study for any reason,
General or specific changes in the patient's condition rendered the patient unacceptable for further treatment in the judgment of the investigator.

Patients were followed for survival every 3 months for two years after removal from study or until death, whichever occurred first. Patients removed from study for unacceptable adverse event(s) were followed until resolution or stabilization of the adverse event.

e. Dose Delays and Modifications

The following table summarizes dose levels available for protocol-specified dose reductions of ficlatuzumab and/or cetuximab (Table 1).

TABLE 1
Dose Reduction Levels

| Dose Level | Ficlatuzumab | Cetuximab |
|---|---|---|
| −2 | 10 mg/kg/q2 weeks | 300 mg/m²/q2 weeks |
| −1 | 15 mg/kg/q2 weeks | 400 mg/m²/q2 weeks |

While serious (Grade 3-4) myelosuppression was not observed with ficlatuzumab monotherapy or the combination of ficlatuzumab with EGFR-inhibitors during phase I development, dose modifications are specified in Table 2A for neutropenia or thrombocytopenia if observed. As cetuximab does not cause myelosuppression, the cetuximab dose was not be affected by any observed neutropenia or thrombocytopenia.

TABLE 2A
Ficlatuzumab and Cetuximab Dose Modifications for Hematologic
Toxicity

| NCI CTCAE Toxicity Grade | | Cetuximab |
|---|---|---|
| (CTCAE v. 4) | Ficlatuzumab Dose | Dose |
| Neutropenia | | |
| Grade 1-2 | Maintain dose level | Maintain |
| 1 (1500-1999/mm³) | | dose level |
| 2 (1000-1499/mm³) | | |
| Grade ≥ 3: | Hold dose. When | Maintain |
| 3 (500-999/mm³ | recovered to Grade 2 or | dose level |
| 4 (<500/mm³) | better, decrease by 1 dose | |
| | level (i.e, reduce to | |
| | 15 mg/kg every two weeks). | |
| Thrombocytopenia | | |
| Grade 1 (75,000/mm³⁻LLN) | Maintain dose level | Maintain |
| | | dose level |
| Grade ≥ 2: | Hold dose. Re-assess prior | Maintain |
| 2 (50,000-74,999/mm³) | to next scheduled dose. | dose level |
| 3 (25,000-49,999/mm³) | When recovered to Grade 1 | |
| 4 (<25,000/mm³) | or better, decrease by 1 | |
| | dose level (i.e, reduce to | |
| | 15 mg/kg every two weeks). | |

Adverse events observed and deemed to be at least possibly related to study drug(s) were managed according to the guidelines for dose interruption, delay, or reduction.

If the toxicity was clearly and solely attributed to ficlatuzumab, the ficlatuzumab was withheld or reduced as described below in Table 2B. If the toxicity was at least possibly related to both ficlatuzumab and cetuximab, then both study drugs were modified as described below.

TABLE 2B
Ficlatuzumab and Cetuximab Dose Reductions for Non-Hematologic
Toxicity

| NCI CTCAE Toxicity Grade | | |
|---|---|---|
| (CTCAE v. 4) | Ficlatuzumab Dose | Cetuximab Dose |
| Metabolic^{a} | | |
| Hypomagnesemia, | | |
| Hypokalemia, | | |
| Hypophosphatemia, or | | |
| Hyponatremia | | |
| Grade ≥ 3, Asymptomatic | Hold drug. Administer PO | Hold drug. Administer PO |
| | or IV replacement and | or IV replacement and re- |
| | reassess. When Grade ≤ 2, | assess. When Grade ≤ 2, |
| | continue drug at same dose | continue drug at same dose |
| | level. Same day re- | level. Same day re- |
| | assessment and dosing is | assessment and dosing is |
| | permissible. | permissible. |
| Grade ≥ 3, Symptomatic | Hold drug. Administer PO | Hold drug. Administer PO |
| | or IV replacement until | or IV replacement until |
| | ≤Grade 2 and asymptomatic, | ≤Grade 2 and asymptomatic, |
| | then reduce by one dose | then reduce by one dose |
| | level. Same day re- | level. Same day re- |
| | assessment and dosing is | assessment and dosing is |
| | permissible. | permissible. |
| Hepatic Function | | |
| AST and/or ALT Elevation | | |
| Grade 2 | Reduce by one dose level | Reduce by one dose level |
| Grade ≥ 3 | Hold drug until ≤grade 2 | Hold drug until ≤grade 2 |
| | then reduce by one dose | then reduce by one dose |
| | level (i.e, reduce to | level (i.e, reduce to |
| | 15 mg/kg every two weeks) | 15 mg/kg every two weeks) |
| AST or ALT elevation >3 × ULN | Discontinue ficlatuzumab | Discontinue ficlatuzumab |
| and concomitant elevation of | | |
| bilirubin >2 × ULN | | |
| Low Albumin | | |
| Grade 3 | No dose reduction | No dose reduction |
| Grade 4 | Reduce by one dose level | No dose reduction |
| Edema^{b} | | |
| Facial/Neck Edema | | |
| Grade 2 (intolerable) or | Hold drug. Administer | No dose reduction |
| Grade ≥ 3 | steroids and/or PO or IV | |
| | diuretics as clinically | |
| | indicated. Resume drug | |
| | when ≤grade 2 and reduce | |
| | by one dose level (i.e, | |
| | reduce to 15 mg/kg every | |
| | two weeks) | |
| Peripheral Edema | | |
| Grade 2 | No dose reduction. Treat | |
| | with PO diuretics as | |
| | clinically indicated. | |
| Grade ≥ 3 | Hold drug and administer | |
| | PO or IV diuretics as | |
| | clinically indicated. | |
| | Resume drug when ≤grade 2 | |
| | and reduce by one dose | |
| | level (i.e, reduce to | |
| | 15 mg/kg every two weeks). | |
| Fatigue | | |
| Grade ≥ 3, lasting more than 7 | Hold drug until ≤grade 2 | Hold drug until ≤grade 2 |
| days | then reduce by one dose | then reduce by one dose |
| | level (i.e, reduce to | level (i.e., 400 mg/m²/ |
| | 15 mg/kg every two weeks) | every two weeks) |
| Nausea/Vomiting | | |
| ≥Grade 3 with maximal | Hold drug until ≤grade 2 | Hold drug until ≤grade 2 |
| medical management | then reduce by one dose | then reduce by one dose |
| | level (i.e, reduce to | level (i.e., 400 mg/m²/ |
| | 15 mg/kg every two weeks) | every two weeks) |
| Rash, Acneiform | | |
| ≥Grade 3 | Maintain dose level | First Occurrence: Hold |
| | | drug for two weeks. If |
| | | improved to ≤grade 2 then |
| | | restart cetuximab at same |
| | | dose level. If rash remains |
| | | ≥Grade 3 then discontinue |
| | | cetuximab. |
| | | Second Occurrence: Hold |
| | | drug for two weeks. If |
| | | improved to ≤grade 2 then |
| | | restart cetuximab at dose |
| | | level-1 (i.e., 400 mg/m²/ |
| | | every two weeks). If rash |
| | | remains ≥Grade 3 then |
| | | discontinue cetuximab. |
| | | Third Occurrence: Hold |
| | | drug for two weeks. If |
| | | improved to ≤grade 2 then |
| | | restart cetuximab at dose |
| | | level-2 (i.e., 300 mg/m²/ |
| | | every two weeks). If rash |
| | | remains ≥Grade 3 then |
| | | discontinue cetuximab. |
| | | Fourth occurrence: |
| | | discontinue cetuximab. |
| Other, Grade ≥ 3 | Hold drug until ≤grade 1 | Hold drug until ≤grade 1 |
| | or baseline, then reduce by | or baseline, then reduce by |
| | one dose level (i.e, reduce | one dose level (i.e., |
| | to 15 mg/kg every two | 400 mg/m²/every two |
| | weeks). | weeks). |

^{a}Cetuximab and ficlatuzumab can be associated with electrolyte abnormalities including hypomagnesemia and hypokalemia. Supplemental oral or IV electrolytes should be administered as indicated by the treating investigator. Sustained repletion may require both chronic oral and IV replacement. An EKG is strongly recommended in the event of: 1) symptomatic Grade ≥ 3 hypomagnesemia or hypokalemia or 2) asymptomatic Grade 4 hypomagnesemia or hypokalemia. Interventions, including hospitalization as necessary for correction of electrolyte derangement, should occur in accordance with clinical severity and investigator judgment.
^{b}Ficlatuzumab can be associated with edema. In patients with HNSCC, therapeutics that cause peripheral edema can also be associated with facial/neck edema. In the case of intolerable grade 2 or grade ≥ 3 facial/neck edema, hold ficlatuzumab. A brief steroid pulse (eg. Prednisone 40 mg/daily for 5 days) may also be considered. In the case of intolerable grade 2 or grade ≥ 3 peripheral edema, hold ficlatuzumab and administer PO or IV diuretics as indicated.

Subjects were permitted up to two dose reductions. Subjects at the lowest ficlatuzumab dose level (10 mg/kg) who experience an attributable Grade 3 or 4 toxicity were discontinued from ficlatuzumab. However, if in the investigator's opinion there was evidence of clinical benefit, a subject's treatment resumed at 10 mg/kg after the AE was resolved or ameliorated to ≤Grade 2 or baseline.

If any observed toxicity at least possibly related to ficlatuzumab and/or cetuximab prevented dosing within the scheduled study visit window, the dose for that study visit was skipped and the next study drug administration occurred at the next scheduled dose. If a ficlatuzumab-related toxicity resulted in two consecutive missed doses, the ficlatuzumab was discontinued permanently. However, if in the investigator's opinion there was evidence of clinical benefit, a subject with two consecutive missed doses was permitted to resume treatment after the AE had resolved to the minimum specifications in Table 2A-2B.

Cetuximab-related dermatologic toxicity was be graded according to the criteria outlined in Table 3 below. According to physician judgment, if a patient experienced ≥grade 3 rash, cetuximab treatment was adjusted according to Tables 2A-B above. In patients with mild and moderate skin adverse events, cetuximab continued without adjustment.

TABLE 3
Grading of Cetuximab-Related Skin Changes

| | 1 | 2 | 3 | 4 |
|---|---|---|---|---|
| Pruritus* | Mild or | Intense or | Intense or | — |
| | localized | widespread | widespread and | |
| | | | interfering with | |
| | | | ADL | |
| Rash/acneiform* | Papules and/or | Papules and/or | Papules and/or | Papules |
| | pustules | pustules covering | pustules covering | and/or |
| | covering <10% | 10-30% BSA, | >30% BSA, | pustules |
| | BSA, which | which may or may | which may or | covering any |
| | may or may | not be associated | may not be | % BSA, |
| | not be | with symptoms of | associated with | which may or |
| | associated with | pruritus or | symptoms of | may not be |
| | symptoms of | tenderness; | pruritus or | associated |
| | pruritus or | associated with | tenderness; | with |
| | tenderness | psychosocial | limiting self-care | symptoms of |
| | | impact; limiting | ADL; associated | pruritus or |
| | | instrumental ADLI; | with local | tenderness |
| | | Responds promptly | superinfection | and are |
| | | to symptomatic | with oral | associated |
| | | treatment | antibiotics | with extensive |
| | | | indicated; | superinfection |
| | | | Prolonged. | with IV |
| | | | | antibiotics |
| | | | | indicated; |
| | | | | life |
| | | | | threatening |
| | | | | consequences |
| Paronychia* | Nail fold | Localized | Surgical | — |
| | edema or | intervention | intervention or IV | |
| | erythema; | indicated; oral | antibiotics | |
| | disruption of | intervention | indicated; | |
| | the cuticle | indicated (e.g., | limiting self | |
| | | antibiotic, | care ADL | |
| | | antifungal, | | |
| | | anti viral); | | |
| | | nail fold edema or | | |
| | | erythema with | | |
| | | pain; associated | | |
| | | with discharge or | | |
| | | nail plate | | |
| | | separation; limiting | | |
| | | instrumental ADL | | |

*Onset of grade 3 will require dose modification for cetuximab. See Tables 3A-B above.

Skin rash, ranging from dry skin and erythema to a pustular eruption is extremely common during cetuximab therapy. Biopsy of the papulopustular rash demonstrates histopathologic suppurative inflammation and not acne vulgaris. Although the initial rash is sterile, superinfection may occur.

Patients developing dermatologic AEs while receiving cetuximab were monitored for the development of inflammatory or infectious sequelae, and appropriate treatment of these symptoms initiated.

Management of reactions to cetuximab infusion was performed as described in Table 4.

TABLE 4
Management of Cetuximab Infusion Reaction

| Grade | Management^{a} |
|---|---|
| Grade 1: | For mild infusion reactions manifesting only as delayed drug fever, |
| Transient flushing | consider administering prophylactic antihistamine medications for |
| or rash; drug fever | subsequent doses. Maintain the cetuximab dose but slow the infusion |
| <38° C. (<100.4° F.) | rate by 50%. Acetaminophen or a non-steroidal anti-inflammatory drug |
| | (NSAID) may be administered prior to subsequent cetuximab infusions, |
| | if not otherwise contraindicated in subjects. |
| Grade 2: | For moderate infusion reactions manifesting only as delayed drug fever, |
| Rash; flushing; | slow the infusion rate for cetuximab by 50%, and consider |
| urticaria; dyspnea; | administration of antihistamine medications and/or steroidal |
| drug fever | medications. Maintain the cetuximab dose. Acetaminophen or a non- |
| ≥38° C. (≥100.4° F.) | steroidal anti-inflammatory drug (NSAID) may be administered prior to |
| | subsequent cetuximab infusions, if not otherwise contraindicated in |
| | subjects. |
| Grade 3: | Severe infusion reactions requires immediate interruption of |
| Symptomatic | cetuximab infusion and permanent discontinuation from further |
| bronchospasm with | treatment with cetuximab. Appropriate medical therapy including |
| or without urticaria; | epinephrine, corticosteroids, diphenhydramine, bronchodilators, and |
| parenteral | oxygen should be available for use in the treatment of such reactions. |
| medication(s) | Subjects should be carefully observed until the complete resolution of |
| indicated; allergy- | all signs and symptoms. |
| related | |
| edema/angioedema; | |
| hypotension | |
| Grade 4: | NO FURTHER CETUXIMAB |
| Anaphylaxis | Life-threatening infusion reactions require immediate interruption of |
| | cetuximab infusion and permanent discontinuation from further |
| | treatment with cetuximab. Appropriate medical therapy including |
| | epinephrine, corticosteroids, diphenhydramine, bronchodilators, and |
| | oxygen should be available for use in the treatment of such reactions. |
| | Subjects should be carefully observed until the complete resolution of |
| | all signs and symptoms. |

^{a}Study Therapy Retreatment Following Infusion Reactions: Once a cetuximab infusion rate has been decreased due to an infusion reaction, it will remain decreased for all subsequent infusions. If the subject has a second infusion reaction with the slower infusion rate, the infusion should be stopped, and the subject should receive no further cetuximab treatment. If a subject experiences a Grade 3 or 4 infusion reaction at any time, the subject should receive no further cetuximab treatment.

In the event of acute onset (grade≥2) or worsening pulmonary symptoms which are not thought to be related to underlying cancer, both cetuximab and ficlatuzumab was interrupted and a prompt investigation of these symptoms occurred. Neither ficlatuzumab nor cetuximab retreatment occurred until these symptoms resolved to grade 1. If interstitial lung disease was confirmed, both cetuximab and ficlatuzumab were discontinued permanently and the patient was treated appropriately.

f. Response Assessment

For the purposes of this study, patients were re-evaluated for response every 8 weeks.

Response and progression were evaluated in this study using the new international criteria proposed by the revised Response Evaluation Criteria in Solid Tumors (RECIST) guideline (version 1.1) (Eisenhauer, E. A. et al. Eur J Cancer (2009) 45:228-247). Changes in the largest diameter (unidimensional measurement) of the tumor lesions and the shortest diameter in the case of malignant lymph nodes were used.

### Malignant Disease Evaluation

To assess objective response, it was necessary to estimate the overall tumor burden at baseline to which subsequent measurements were compared. Measurable disease is defined by the presence of at least one measurable lesion.

All measurements were recorded in metric notation by use of a ruler or calipers. The same method of assessment and the same technique was used to characterize each identified lesion at baseline and during follow-up. All baseline evaluations were performed as closely as possible to the beginning of treatment and never more than four weeks before registration.

At baseline, the primary tumor and pathologic neck lymph nodes were characterized as either measurable or non-measurable.

Measurable primary tumors are lesions that can be accurately measured in at least one dimension (longest diameter to be recorded) as ≥20 mm (2.0 cm) with conventional techniques or as ≥10 mm (1.0 cm) with spiral CT scan.

Neck lymph nodes were considered pathologic and measurable if short axis ≥15 mm. Neck lymph nodes were considered pathologic but non-measurable if short axis ≥10 mm but <15 mm. Neck lymph nodes were considered non-pathologic and non-measurable if short axis <10 mm.

All other lesions, including small lesions [longest diameter <20 mm (2.0 cm) with conventional techniques or <10 mm (1.0 cm) with spiral CT scan] are truly non-measurable lesions.

Lesions considered to be truly non-measurable include the following: bone lesions, leptomeningeal disease, ascites, pleural/pericardial effusion, inflammatory breast disease, lymphangitis cutis/pulmonis, abdominal masses that are not confirmed and followed by imaging techniques, and cystic lesions.

All measurable lesions up to a maximum of two lesions per organ and five lesions in total, representative of all involved organs, were identified as target lesions and recorded and measured at baseline. Target lesions were selected on the basis of their size (lesions with the longest diameter), were representative of all involved organs, and lent themselves to reproducible repeated measurements. If the largest lesion did not lend itself to reproducible measurement in which circumstance the next largest lesion which can be measured reproducibly was selected. A sum of the diameters (longest for non-nodal lesions, short axis for nodal lesions) for all target lesions was calculated and reported as the baseline sum diameters. If lymph nodes were to be included in the sum, then only the short axis was added into the sum. The baseline sum diameters were used as reference to further characterize any objective tumor regression in the measurable dimension of the disease.

The sum of the longest diameter of the primary tumor, and the short axis diameter of target pathologic lymph nodes, were calculated at baseline and reported as the baseline sum diameter. All other lesions (or sites of disease) including any measurable lesions over and above the five target lesions were identified as non-target lesions and were recorded at baseline. Measurements of these lesions were not required, but the presence, absence, or in rare cases unequivocal progression of each were noted throughout follow-up.

### Response Criteria

For the evaluation of target lesions, a complete response (CR) was the disappearance of all target lesions. Any pathological lymph nodes (whether target or non-target) must have had reduction in short axis to <10 mm. A partial response (PR) was at least a 30% decrease in the sum of target lesion diameters (longest diameter of non-nodal lesions; short axis diameter of the target lymph nodes), taking as reference the baseline sum diameter. Progressive disease (PD) was at least a 20% increase in the sum of target lesion diameters (longest diameter of non-nodal lesions; short axis diameter of the target lymph nodes), taking as reference the smallest sum diameter recorded since the baseline sum diameter measurements. In addition to the relative increase of 20%, the sum needed to demonstrate an absolute increase of at least 5 mm. The appearance of one or more new lesions was also considered progression. Stable disease (SD) was neither sufficient shrinkage to qualify for PR nor sufficient increase to qualify for PD, taking as reference the smallest sum diameters while on study.

For evaluation of nontarget lesions (all other lesions or sites of disease, not required but should be noted) a complete response (CR) was the disappearance of all nontarget lesions. A partial response/stable disease (SD) was the persistence of one or more nontarget lesion(s). Progressive disease (PD) was the appearance of one or more new lesions and/or unequivocal progression of existing non-target lesions. Unequivocal progression did not normally trump target lesion status. It must be representative of overall disease status change, not a single lesion increase. Although a clear progression of “non-target” lesions only is exceptional, the opinion of the treating physician prevailed in such circumstances, and the progression status was confirmed at a later time by the review panel (or Principal Investigator).

Patients with a global deterioration of health status requiring discontinuation of treatment without objective evidence of disease progression at that time were classified as having symptomatic deterioration.

The best overall response was the best response recorded from registration until disease progression/recurrence, taking as reference for progressive disease the smallest measurements recorded since registration. Table 5 below provides overall responses for all possible combinations of tumor responses in target and nontarget lesions, with or without new lesions.

To be assigned a status of stable disease, measurements needed to meet the stable disease criteria at least once after study entry at a minimum interval of eight weeks.

TABLE 5
Evaluation for Patients with Measurable Disease (i.e., Target Disease)

| | | | | Best Overall Response |
|---|---|---|---|---|
| Target | Non-Target | New | Overall | when Confirmation is |
| Lesions | Lesions | Lesions | Response | Required* |
| CR | CR | No | CR | ≥4 wks. |
| | | | | Confirmation** |
| CR | Non-CR/Non-PD | No | PR | ≥4 wks. |
| CR | Not evaluated | No | PR | Confirmation** |
| PR | Non-CR/Non-PD/ | No | PR | |
| | not evaluated | | | |
| SD | Non-CR/Non-PD/ | No | SD | Documented at |
| | not evaluated | | | least once ≥4 wks. |
| | | | | from baseline** |
| PD | Any | Yes or | PD | no prior SD, |
| | | No | | PR or CR |
| Any | PD*** | Yes or | PD | |
| | | No | | |
| Any | Any | Yes | PD | |

*See RECIST 1.1 manuscript for further details on what is evidence of a new lesion.
**Only for non-randomized trials with response as primary endpoint.
***In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.
CR = complete response; PR = partial response; SD = stable disease; PD = progressive disease

The first documentation of response was the time between initiation of therapy and first documentation of PR or CR.

To be assigned a status of complete or partial response, changes in tumor measurements needed to be confirmed by repeat assessments performed no less than four weeks after the criteria for response were first met.

Duration of overall response was the period measured from the time that measurement criteria were met for complete or partial response (whichever status was recorded first) until the first date that recurrent or progressive disease was objectively documented, taking as reference the smallest measurements recorded since treatment started.

Duration of overall complete response was the period measured from the time measurement criteria were met for complete response until the first date that recurrent disease was objectively documented.

Duration of stable disease was a measurement from registration until the criteria for disease progression was met, taking as reference the smallest measurements recorded since registration. To be assigned a status of stable disease, measurements must have met the stable disease criteria at least once after study entry at a minimum interval of six weeks.

Survival was measured from the date of entry on study.

Time to progression and progression-free survival was measured from the date of entry on the study to the appearance of new metastatic lesions or objective tumor progression.

Progression-free survival (PFS) was calculated from treatment initiation to disease progression or death from any cause.

g. Drug Information

Ficlatuzumab Concentrate for Injection, 20 mg/mL, was formulated in 10 mM histidine buffer pH 5.8. The formulation also includes 142 mM arginine (for isotonicity) and 0.01% polysorbate 80. The product was sterile filtered and aseptically filled into washed and depyrogenated 5 mL glass vials. An excess fill was provided in the vial to ensure that the label fill of 4.0 mL could be withdrawn. The product was a clear to slightly opalescent, colorless to slightly yellow, solution.

Ficlatuzumab Concentrate for Injection was administered by IV infusion as an admixture with normal saline solution. The admixture solution in an IV bag was connected to an infusion set containing a 0.22 μm low protein-binding in line filter. The IV bag and the infusion set containing the in line filter were shown to be compatible with the admixture. The filtered admixture solution was clear to slightly opalescent.

Ficlatuzumab was stored under refrigerated conditions (2° C.-8° C.) and in a secure location.

Cetuximab is an anti-EGFR receptor humanized chimeric monoclonal antibody. Cetuximab was expressed in SP2/0 myeloma cell line, grown in large scale cell culture bioreactors, and purified to a high level purity using several purification steps including protein A chromatography, ion exchange chromatography, low pH treatment, and nanofiltration. Cetuximab is not known to be a vesicant.

Preparation and Administration: Cetuximab was not administered as an IV push or bolus. Cetuximab was administered with the use of a low protein binding 0.22-micrometer in-line filter.

Cetuximab was supplied as a 50-mL, single-use vial containing 100 mg of cetuximab at a concentration of 2 mg/mL in phosphate buffered saline. The solution was clear and colorless and could contain a small amount of easily visible white amorphous cetuximab particulates.

Cetuximab was administered via infusion pump or syringe pump. Cetuximab was piggybacked to the patient's infusion line.

Following the cetuximab infusion, a one-hour observation period was recommended.

### 2. Results

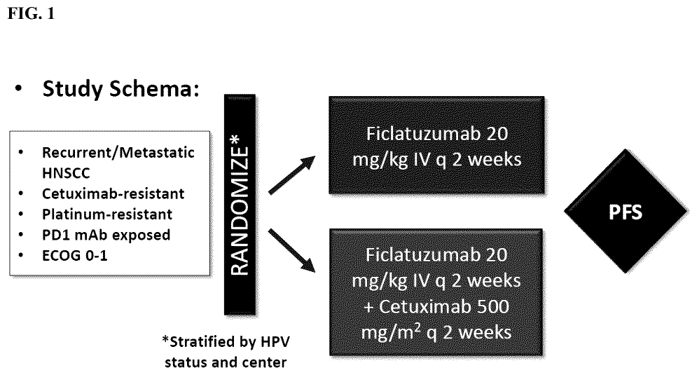

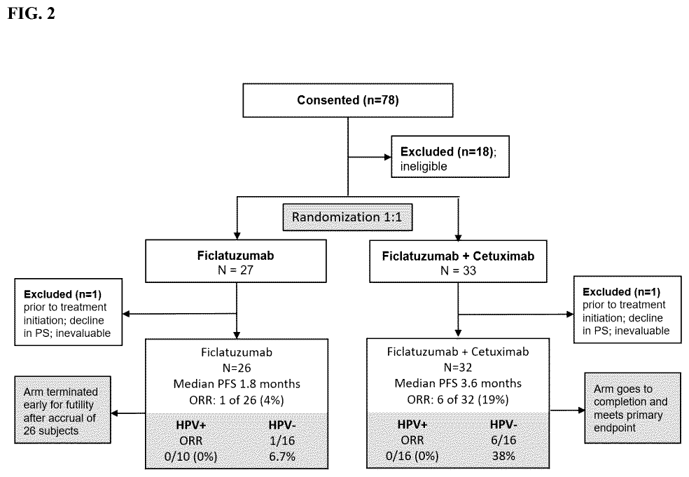

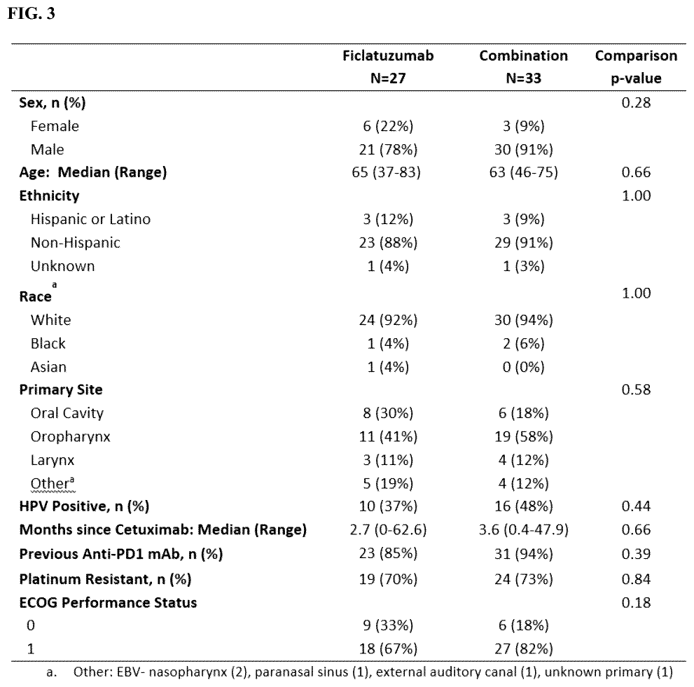

A total of 60 patients were randomized and 58 initiated study treatment from January 2018 to December 2020 as depicted FIG. 1 and FIG. 2. The baseline characteristics of the study population were balanced across the two treatment arms (FIG. 3). Per design, all patients were cetuximab-resistant; median time since prior cetuximab exposure was 2.7 and 3.6 months on the ficlatuzumab and combination arms, respectively. Fifty-four of 60 (90%) had progressed on anti-PD1 mAb and 43 (72%) were platinum-resistant. Ten of 60 (17%) were racial or ethnic minorities under-represented in clinical research.

### Ficlatuzumab Monotherapy Arm

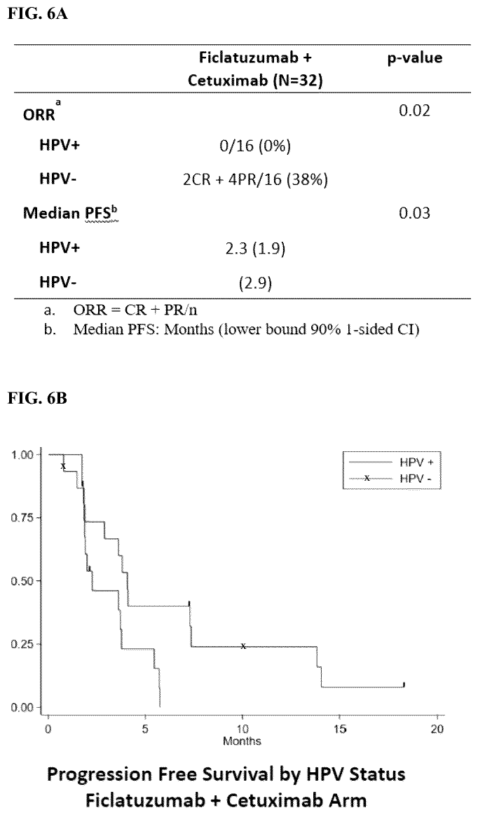

The ficlatuzumab monotherapy arm was stopped for futility after 26 evaluable subjects accrued a median progression free survival (mPFS) of 1.8 months (lower bound 90% CI: 1.7 months), median OS was 4.9 months (lower bound 90% CI, 3.3 months), and an overall response rate (ORR) of 1/26 (4%) with the one response being a partial response in an HPV negative subject, as shown in FIGS. 6A and 6B.

### Ficlatuzumab and Cetuximab Combination Arm

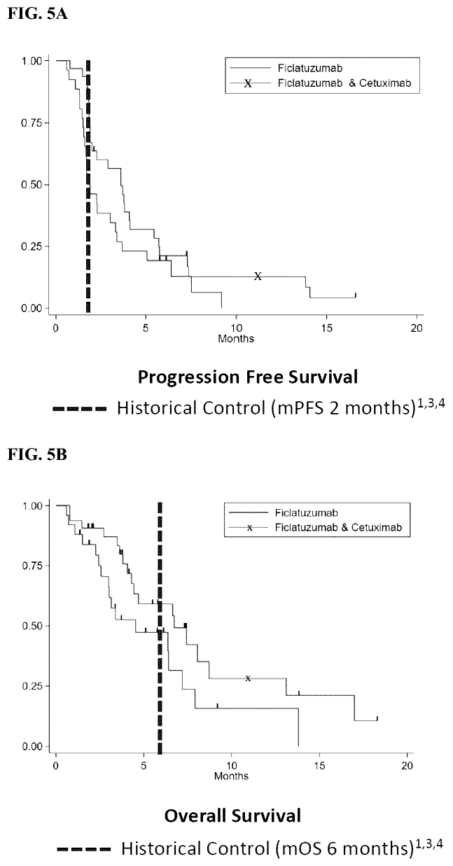

The ficlatuzumab and cetuximab combination arm completed accrual with 32 evaluable subjects and met the primary endpoint. Subjects in the ficlatuzumab and cetuximab combination arm accrued mPFS of 3.6 months (lower bound 90% CI, 2.3 months; p=0.04; FIG. 5A), meeting protocol-specified criteria for phase III study. The median OS was 7.3 months (lower bound 90% CI, 4.8 months; FIG. 5B). The ORR was 6/32 (19%), including two complete (CR) and four partial responses (PR). One patient with CR died from unrelated cardiovascular disease while still in CR. The second patient remained in CR 46 months after initiating protocol treatment and was on ficlatuzumab only since month 18 due to recurrent, severe acneiform rash despite dose reduction of cetuximab. As shown by the Kaplan-Meier curves in FIGS. 5A PFS, all patients receiving ficlatuzumab had progressed by around 9 months, whereas some ficlatuzumab/cetuximab subjects had still not progressed beyond 15 months. Similar trends were shown in FIG. 5B comparing the control and treatment arms. The data suggest the probability of a positive outcome is greater for subjects receiving the combination treatment.

Of note, all objective responses occurred in patients with HPV-negative disease. Due to the unexpectedly high response rate in pan-refractory, HPV-negative disease, a post hoc analysis was performed in the HPV-stratified subgroups on the combination arm. As shown in FIG. 6A, the ORR in HPV-positive vs. HPV-negative patients was 0/16 (0%) vs. 6/16 (38%), p=0.02. The median PFS was 2.3 vs. 4.1 months, p=0.03 (FIG. 6B). A sensitivity analysis for ORR was performed in HPV-negative patients, excluding patients who had never received platinum due to medical ineligibility; the ORR was 5 of 13 (38%).

In particular, the ORR in HPV-positive subjects was 0% as no HPV-positive subjects were responders, whereas the ORR in HPV-negative subjects was 38% with 2 complete responses and 4 partial responses being recorded amongst the 16 HPV-negative patients (p=0.02). The mPFS of HPV-positive subjects was 2.3 months compared to 4.1 months for HPV-negative subjects had superior ORR (p=0.02) and mPFS (p=0.03). Further, as shown by the Kaplan-Meier curve in FIG. 6B, all HPV-positive subjects had progressed by around 6 months, whereas at around 18 months, some subjects remained progression free. This is a surprising result to have any HPV-negative HNSCC subjects survive this long, given the extremely poor prognosis for these patients based on the currently available standard of care.

These results are significant as treatment of HNSCC with cetuximab provides only a minor benefit in these cancers, with an ORR of 10-13%. Accordingly, among other things, the present disclosure provides compositions and therapeutic regimens for subjects with HNSCC that are HPV-negative that yield enhanced clinical outcomes compared to current standards of treatments, e.g., immunotherapy (such as immune-checkpoint inhibitors like pembrolizumab) with platinum therapy, or cetuximab.

The data clearly indicate that HPV-negative subjects experienced superior outcomes compared to HPV-positive subjects and support a strong therapeutic benefit in HPV-negative subjects to this combination therapy. This was surprising as HPV-positive patients generally fare better than HPV-negative patients with traditional treatments for HNSCC, with HPV-positive status being a positive prognostic indicator. Accordingly, the discovery that HPV-negative status would be a positive prognostic indicator for treatment with cetuximab and ficlatuzumab in recurrent/metastatic HNSCC, whereas HPV-positive status would be a negative prognostic indicator is entirely surprising and unexpected.

Accordingly, the data suggest that subjects with HPV-negative recurrent or metastatic HNSCC could greatly benefit from treatment with than cetuximab and ficlatuzumab comprised with cetuximab alone as a standard of care for second line therapy or later line therapy in subjects with immune checkpoint-resistant and/or platinum resistant HNSCC.

### Toxicity

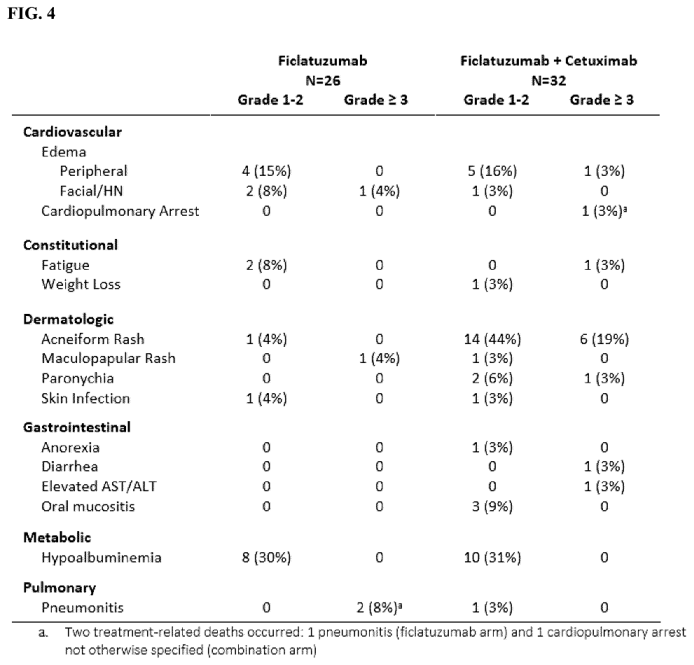

Safety data, including treatment-emergent AEs attributed to ficlatuzumab and/or cetuximab, are summarized in FIG. 4. On the monotherapy arm, the most common AEs were hypoalbuminemia (30%) and edema (27%), expected class toxicities for HGF/cMet inhibitors. On the combination arm, the most common toxicities were acneiform rash (63%), a class toxicity for EGFR inhibitors, hypoalbuminemia (31%), and edema (22%). Three cases of pneumonitis, a rare but known class toxicity of HGF/cMet inhibitors, were observed: two on the monotherapy arm (including one fatal AE) and one on the combination arm.

FIG. 4 provides a detailed breakdown of the types of toxicities observed grouped by Grade 1-2 and ≥Grade 3. In the ficlatuzumab arm, Grade 1-2 edema, fatigue, acneiform rash, skin infection, and hypalbuminemia were observed, along with Grade or greater edema, maculopapular rash and pneumonitis. In the combination arm, Grade 1-2 toxicities observed include edema, weight loss, various dermatologic toxicities, anorexia, mucositis, hypoalbuminemia, and pneumonitis, with some Grade 3 or greater edema, fatigue, and dermatologic and gastrointestinal toxicities. The data show that the combination therapy of ficlatuzumab and cetuximab was well tolerated with expected class toxicities from HGF/c-MET inhibitors including common adverse events edema and hypoalbuminemia and uncommon adverse event pneumonitis.

### Example 2—Phase III Trial to Evaluate Cetuximab and Ficlatuzumab in Subjects with Recurrent or Metastatic HNSCC that is HPV-Negative

A phase III trial to evaluate treatment of subjects with recurrent or metastatic HNSCC that is HPV-negative is conducted. Subjects with recurrent or metastatic HNSCC that is determined to be HPV-negative and that is platinum-resistant or platinum-ineligible and/or immune checkpoint-inhibitor-resistant or immune checkpoint inhibitor ineligible are randomized into two study groups: a control arm that received cetuximab at a dose of 500 mg/m² every two weeks and a treatment arm that receives cetuximab at a dose of 500 mg/m² with 20 mg/kg ficlatuzumab every two weeks by concurrent i.v. administration.

Subject remain in the study until progression, death, or withdrawal from the study for any reason including experiencing unacceptable toxicity.

Based in part from the results from the Phase II study in Example 1, the results are expected to show that median progression free survival and overall survival are both statistically significantly longer for the treatment arm than the control arm. The results are also expected to show that subjects with HNSCC that is HPV-negative and also platinum-resistant or platinum-ineligible and/or immune checkpoint-inhibitor-resistant or immune checkpoint inhibitor ineligible experience a greater clinical benefit from treatment with cetuximab and ficlatuzumab rather than cetuximab alone. Safety data are expected to show that the ficlatuzumab and cetuximab in combination are well tolerated with acceptable class toxicities.

SEQUENCE LISTING

| SEQ | | |
|---|---|---|
| ID | | |
| NO: | Description | Sequence |
| 1 | Ficlatuzumab | TYWMH |
| | CDRH1 | |
| 2 | Ficlatuzumab | EINPTNGHTNYNQKFQG |
| | CDRH2 | |
| 3 | Ficlatuzumab | NYVGSIFDY |
| | CDRH3 | |
| 4 | Ficlatuzumab | KASENVVSYVS |
| | CDRL1 | |
| 5 | Ficlatuzumab | GASNRES |
| | CDRL2 | |
| 6 | Ficlatuzumab | GQSYNYPYT |
| | CDRL3 | |
| 7 | Ficlatuzumab | QVQLVQPGAE VKKPGTSVKL SCKASGYTFT |
| | heavy chain | TYWMHWVRQA PGQGLEWIGE INPTNGHTNY NQKFQGRATL |
| | variable | TVDKSTSTAY MELSSLRSED TAVYYCARNY VGSIFDYWGQ |
| | region | GTLLTVSS |
| 8 | Ficlatuzumab | DIVMTQSPDS LAMSLGERVT LNCKASENVV SYVSWYQQKP |
| | light chain | GQSPKLLIYG ASNRESGVPD RFSGSGSATD FTLTISSVQA |
| | variable | EDVADYHCGQ SYNYPYTFGQ GTKLEIK |
| | region | |
| 9 | Cetuximab | NYGVH |
| | CDRH1 | |
| 10 | Cetuximab | VIWSGGNTDYNTPFTS |
| | CDRH2 | |
| 11 | Cetuximab | ALTYYDYEFAY |
| | CDRH3 | |
| 12 | Cetuximab | RASQSIGTNIH |
| | CDRL1 | |
| 13 | Cetuximab | YASESIS |
| | CDRL2 | |
| 14 | Cetuximab | QQNNNWPTT |
| | CDRL3 | |
| 15 | Cetuximab | QVQLKQSGPG LVQPSQSLSI TCTVSGFSLT NYGVHWVRQS |
| | heavy chain | PGKGLEWLGV IWSGGNTDYN TPFTSRLSIN KDNSKSQVFF |
| | variable | KMNSLQSNDT AIYYCARALT YYDYEFAYWG QGTLVTVSA |
| | region | |
| 16 | Cetuximab | DILLTQSPVI LSVSPGERVS FSCRASQSIG TNIHWYQQRT |
| | light | NGSPRLLIKY ASESISGIPS RFSGSGSGTD FTLSINSVES |
| | chain | EDIADYYCQQ NNNWPTTFGA GTKLELKR |
| | variable | |
| | region | |
| 17 | Ficlatuzumab | QVQLVQPGAE VKKPGTSVKL SCKASGYTFT |
| | heavy chain | TYWMHWVRQA PGQGLEWIGE INPTNGHTNY NQKFQGRATL |
| | | TVDKSTSTAY MELSSLRSED TAVYYCARNY VGSIFDYWGQ |
| | | GTLLTVSSAS TKGPSVFPLA PSSKSTSGGT AALGCLVKDY |
| | | FPEPVTVSWN SGALTSGVHT FPAVLQSSGL YSLSSVVTVP |
| | | SSSLGTQTYI CNVNHKPSNT KVDKRVEPKS CDKTHTCPPC |
| | | PAPELLGGPS VFLFPPKPKD TLMISRTPEV TCVVVDVSHE |
| | | DPEVKFNWYV DGVEVHNAKT KPREEQYNST YRVVSVLTVL |
| | | HQDWLNGKEY KCKVSNKALP APIEKTISKA KGQPREPQVY |
| | | TLPPSREEMT KNQVSLTCLV KGFYPSDIAV EWESNGQPEN |
| | | NYKTTPPVLD SDGSFFLYSK LTVDKSRWQQ GNVFSCSVMH |
| | | EALHNHYTQK SLSLSPGK |
| 18 | Ficlatuzumab | DIVMTQSPDS LAMSLGERVT LNCKASENVV SYVSWYQQKP |
| | light chain | GQSPKLLIYG ASNRESGVPD RFSGSGSATD FTLTISSVQA |
| | | EDVADYHCGQ SYNYPYTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 19 | Cetuximab | QVQLKQSGPG LVQPSQSLSI TCTVSGFSLT NYGVHWVRQS |
| | heavy chain | PGKGLEWLGV IWSGGNTDYN TPFTSRLSIN KDNSKSQVFF |
| | | KMNSLQSNDT AIYYCARALT YYDYEFAYWG QGTLVTVSAA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKRVEPK SCDKTHTCPP CPAPELLGGP |
| | | SVFLFPPKPK DTLMISRTPE VTCVVVDVSH EDPEVKFNWY |
| | | VDGVEVHNAK TKPREEQYNS TYRVVSVLTV LHQDWLNGKE |
| | | YKCKVSNKAL PAPIEKTISK AKGQPREPQV YTLPPSREEM |
| | | TKNQVSLTCL VKGFYPSDIA VEWESNGQPE NNYKTTPPVL |
| | | DSDGSFFLYS KLTVDKSRWQ QGNVFSCSVM HEALHNHYTQ |
| | | KSLSLSPGK |
| 20 | Cetuximab | DILLTQSPVI LSVSPGERVS FSCRASQSIG TNIHWYQQRT |
| | light | NGSPRLLIKY ASESISGIPS RFSGSGSGTD FTLSINSVES |
| | chain | EDIADYYCQQ NNNWPTTFGA GTKLELKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 21 | Rilotumumab | IYYWS |
| | CDRH1 | |
| 22 | Rilotumumab | YVYYSGSTNYNPSLKS |
| | CDRH2 | |
| 23 | Rilotumumab | GGYDFWSGYFDY |
| | CDRH3 | |
| 24 | Rilotumumab | RASQSVDSNLA |
| | CDRL1 | |
| 25 | Rilotumumab | GASTRAT |
| | CDRL2 | |
| 26 | Rilotumumab | QQYINWPPIT |
| | CDRL3 | |
| 27 | Rilotumumab | MKHLWFFLLL VAAPRWVLSQ VQLQESGPGL VKPSETLSLT |
| | heavy chain | CTVSGGSISI YYWSWIRQPP GKGLEWIGYV YYSGSTNYNP |
| | variable | SLKSRVTISV DTSKNQFSLK LNSVTAADTA VYYCARGGYD |
| | region | FWSGYFDYWG QGTLVTVSS |
| 28 | Rilotumumab | MEAPAQLLFL LLLWLPDTTG EIVMTQSPAT LSVSPGERAT |
| | light chain | LSCRASQSVD SNLAWYROKP GQAPRLLIYG ASTRATGIPA |
| | variable | RFSGSGSGTE FTLTISSLQS EDFAVYYCQQ YINWPPITFG |
| | region | QGTRLEIK |
| 29 | Rilotumumab | QVQLQESGPG LVKPSETLSL TCTVSGGSIS IYYWSWIRQP |
| | heavy chain | PGKGLEWIGY VYYSGSTNYN PSLKSRVTIS VDTSKNQFSL |
| | | KLNSVTAADT AVYYCARGGY DFWSGYFDYW |
| | | GQGTLVTVSS ASTKGPSVFP LAPCSRSTSE STAALGCLVK |
| | | DYFPEPVTVS WNSGALTSGV HTFPAVLQSS GLYSLSSVVT |
| | | VPSSNFGTQT YTCNVDHKPS NTKVDKTVER KCCVECPPCP |
| | | APPVAGPSVF LFPPKPKDTL MISRTPEVTC VVVDVSHEDP |
| | | EVQFNWYVDG VEVHNAKTKP REEQFNSTFR |
| | | VVSVLTVVHQ DWLNGKEYKC KVSNKGLPAP IEKTISKTKG |
| | | QPREPQVYTL PPSREEMTKN QVSLTCLVKG FYPSDIAVEW |
| | | ESNGQPENNY KTTPPMLDSD GSFFLYSKLT VDKSRWQQGN |
| | | VFSCSVMHEA LHNHYTQKSL SLSPGK |
| 30 | Rilotumumab | EIVMTQSPAT LSVSPGERAT LSCRASQSVD SNLAWYRQKP |
| | light chain | GQAPRLLIYG ASTRATGIPA RFSGSGSGTE FTLTISSLQS |
| | | EDFAVYYCQQ YINWPPITFG QGTRLEIKRT VAAPSVFIFP |
| | | PSDEQLKSGT ASVVCLLNNF YPREAKVQWK VDNALQSGNS |
| | | QESVTEQDSK DSTYSLSSTL TLSKADYEKH KVYACEVTHQ |
| | | GLSSPVTKSF NRGEC |
| 31 | Imgatuzumab | QVQLVQSGAE VKKPGSSVKV SCKASGFTFT DYKIHWVRQA |
| | heavy chain | PGQGLEWMGY FNPNSGYSTY AQKFQGRVTI TADKSTSTAY |
| | | MELSSLRSED TAVYYCARLS PGGYYVMDAW GQGTTVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN |
| | | STYRVVSVLT VLHQDWLNGK EYKCKVSNKA LPAPIEKTIS |
| | | KAKGQPREPQ VYTLPPSRDE LTKNQVSLTC LVKGFYPSDI |
| | | AVEWESNGQP ENNYKTTPPV LDSDGSFFLY SKLTVDKSRW |
| | | QQGNVFSCSV MHEALHNHYT QKSLSLSPG |
| 32 | Imgatuzumab | DIQMTQSPSS LSASVGDRVT ITCRASQGIN NYLNWYQQKP |
| | light chain | GKAPKRLIYN TNNLQTGVPS RFSGSGSGTE FTLTISSLQP |
| | | EDFATYYCLQ HNSFPTFGQG TKLEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| 33 | Necitumumab | QVQLQESGPG LVKPSQTLSL TCTVSGGSIS SGDYYWSWIR |
| | heavy chain | QPPGKGLEWI GYIYYSGSTD YNPSLKSRVT MSVDTSKNQF |
| | | SLKVNSVTAA DTAVYYCARV SIFGVGTFDY WGQGTLVTVS |
| | | SASTKGPSVL PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKRVE PKSCDKTHTC PPCPAPELLG |
| | | GPSVFLFPPK PKDTLMISRT PEVTCVVVDV SHEDPEVKFN |
| | | WYVDGVEVHN AKTKPREEQY |
| | | NSTYRVVSVL TVLHQDWLNG KEYKCKVSNK ALPAPIEKTI |
| | | SKAKGQPREP QVYTLPPSRE EMTKNQVSLT CLVKGFYPSD |
| | | IAVEWESNGQ PENNYKTTPP VLDSDGSFFL YSKLTVDKSR |
| | | WQQGNVFSCS VMHEALHNHY TQKSLSLSPG K |
| 34 | Necitumumab | EIVMTQSPAT LSLSPGERAT LSCRASQSVS SYLAWYQQKP |
| | light chain | GQAPRLLIYD ASNRATGIPA RFSGSGSGTD FTLTISSLEP |
| | | EDFAVYYCHQ YGSTPLTFGG GTKAEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 35 | Amivantamab | QVQLVESGGG VVQPGRSLRL SCAASGFTFS TYGMHWVRQA |
| | heavy chain | PGKGLEWVAV IWDDGSYKYY GDSVKGRFTI SRDNSKNTLY |
| | (A chain) | LQMNSLRAED TAVYYCARDG ITMVRGVMKD |
| | | YFDYWGQGTL VTVSSASTKG PSVFPLAPSS KSTSGGTAAL |
| | | GCLVKDYFPE PVTVSWNSGA LTSGVHTFPA VLQSSGLYSL |
| | | SSVVTVPSSS LGTQTYICNV NHKPSNTKVD KRVEPKSCDK |
| | | THTCPPCPAP ELLGGPSVFL FPPKPKDTLM ISRTPEVTCV |
| | | VVDVSHEDPE VKFNWYVDGV EVHNAKTKPR |
| | | EEQYNSTYRV VSVLTVLHQD WLNGKEYKCK VSNKALPAPI |
| | | EKTISKAKGQ PREPQVYTLP PSREEMTKNQ VSLTCLVKGF |
| | | YPSDIAVEWE SNGQPENNYK TTPPVLDSDG SFLLYSKLTV |
| | | DKSRWQQGNV FSCSVMHEAL HNHYTQKSLS LSPGK |
| 36 | Amivantamab | QVQLVQSGAE VKKPGASVKV SCETSGYTFT SYGISWVRQA |
| | heavy chain | PGHGLEWMGW ISAYNGYTNY AQKLQGRVTM TTDTSTSTAY |
| | (B chain) | MELRSLRSDD TAVYYCARDL RGTNYFDYWG QGTLVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKRVEPK SCDKTHTCPP CPAPELLGGP |
| | | SVFLFPPKPK DTLMISRTPE VTCVVVDVSH EDPEVKFNWY |
| | | VDGVEVHNAK TKPREEQYNS |
| | | TYRVVSVLTV LHQDWLNGKE YKCKVSNKAL PAPIEKTISK |
| | | AKGQPREPQV YTLPPSREEM TKNQVSLTCL VKGFYPSDIA |
| | | VEWESNGQPE NNYKTTPPVL DSDGSFFLYS RLTVDKSRWQ |
| | | QGNVFSCSVM HEALHNHYTQ KSLSLSPGK |
| 37 | Amivantamab | AIQLTQSPSS LSASVGDRVT ITCRASQDIS SALVWYQQKP |
| | light chain | GKAPKLLIYD ASSLESGVPS RFSGSESGTD FTLTISSLQP |
| | (C chain) | EDFATYYCQQ FNSYPLTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 38 | Amivantamab | DIQMTQSPSS VSASVGDRVT ITCRASQGIS NWLAWFQHKP |
| | light chain | GKAPKLLIYA ASSLLSGVPS RFSGSGSGTD FTLTISSLQP |
| | (D chain) | EDFATYYCQQ ANSFPITFGQ GTRLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 39 | Zalutumumab | QVQLVESGGG VVQPGRSLRL SCAASGFTFS TYGMHWVRQA |
| | heavy chain | PGKGLEWVAV IWDDGSYKYY GDSVKGRFTI SRDNSKNTLY |
| | | LQMNSLRAED TAVYYCARDG ITMVRGVMKD |
| | | YFDYWGQGTL VTVSSASTKG PSVFPLAPSS KSTSGGTAAL |
| | | GCLVKDYFPE PVTVSWNSGA LTSGVHTFPA VLQSSGLYSL |
| | | SSVVTVPSSS LGTQTYICNV NHKPSNTKVD KRVEPKSCDK |
| | | THTCPPCPAP ELLGGPSVFL FPPKPKDTLM ISRTPEVTCV |
| | | VVDVSHEDPE VKFNWYVDGV EVHNAKTKPR |
| | | EEQYNSTYRV VSVLTVLHQD WLNGKEYKCK VSNKALPAPI |
| | | EKTISKAKGQ PREPQVYTLP PSREEMTKNQ VSLTCLVKGF |
| | | YPSDIAVEWE SNGQPENNYK TTPPVLDSDG SFFLYSKLTV |
| | | DKSRWQQGNV FSCSVMHEAL HNHYTQKSLS LSPGK |
| 40 | Zalutumumab | AIQLTQSPSS LSASVGDRVT ITCRASQDIS SALVWYQQKP |
| | light chain | GKAPKLLIYD ASSLESGVPS RFSGSESGTD FTLTISSLQP |
| | | EDFATYYCQQ FNSYPLTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 41 | Panitumumab | QVQLQESGPG LVKPSETLSL TCTVSGGSVS SGDYYWTWIR |
| | heavy chain | QSPGKGLEWI GHIYYSGNTN YNPSLKSRLT ISIDTSKTQF |
| | | SLKLSSVTAA DTAIYYCVRD RVTGAFDIWG QGTMVTVSSA |
| | | STKGPSVFPL APCSRSTSES TAALGCLVKD YFPEPVTVSW |
| | | NSGALTSGVH TFPAVLQSSG LYSLSSVVTV PSSNFGTQTY |
| | | TCNVDHKPSN TKVDKTVERKC |
| 42 | Panitumumab | DIQMTQSPSS LSASVGDRVT ITCQASQDIS NYLNWYQQKP |
| | light chain | GKAPKLLIYD ASNLETGVPS RFSGSGSGTD FTFTISSLQP |
| | | EDIATYFCQH FDHLPLAFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 43 | Nimotuzumab | LSSLRSEDTA FYFCTRQGLW FDSDGRGFDF WGQGTTVTVS |
| | heavy chain | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKKVP |
| 44 | Nimotuzumab | DIQMTQSPSS LSASVGDRVT ITCRSSQNIV HSNGNTYLDW |
| | light chain | YQQTPGKAPK LLIYKVSNRF SGVPSRFSGS GSGTDFTFTI |
| | | SSLQPEDIAT YYCFQYSHVP WTFGQGTKLQ ITREVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| 45 | Matuzumab | QVQLVQSGAE VKKPGASVKV SCKASGYTFT |
| | heavy chain | SHWMHWVRQA PGQGLEWIGE FNPSNGRTNY |
| | | NEKFKSKATM TVDTSTNTAY MELSSLRSED TAVYYCASRD |
| | | YDYDGRYFDY WGQGTLVTVS SASTKGPSVF PLAPSSKSTS |
| | | GGTAALGCLV KDYFPEPVTV SWNSGALTSG VHTFPAVLQS |
| | | SGLYSLSSVV TVPSSSLGTQ TYICNVNHKP SNTKVDKKVE |
| | | PKS |
| 46 | Matuzumab | DIQMTQSPSS LSASVGDRVT ITCSASSSVT YMYWYQQKPG |
| | light | KAPKLLIYDT SNLASGVPSR FSGSGSGTDY TFTISSLQPE |
| | chain | DIATYYCQQW SSHIFTFGQG TKVEIKRTVA APSVFIFPPS |
| | | DEQLKSGTAS VVCLLNNFYP REAKVQWKVD NALQSGNSQE |
| | | SVTEQDSKDST YSLSSTLTLS KADYEKHKVY ACEVTHQGLS |
| | | SPVTKSFNRG E |

1. A method of treating recurrent or metastatic head and neck squamous cell carcinoma (HNSCC) in a subject
having recurrent or metastatic HNSCC that is human papillomavirus (HPV) negative,
the method comprising administering to the subject an effective amount of cetuximab with an effective amount of ficlatuzumab, thereby treating the HNSCC that is HPV negative.

2. (canceled)

3. The method of claim 1, wherein HPV status of the HNSCC is determined by p16 immunohistochemistry or by tumoral DNA analysis, wherein the HNSCC is HPV negative when the HNSCC is classified as p16 negative or the tumoral DNA does not comprise HPV DNA.

4-7. (canceled)

8. The method of claim 1, wherein the HNSCC is PD-1 or PD-L1 immunotherapy-resistant or is platinum-resistant, or wherein the subject is ineligible for platinum chemotherapy.

9. (canceled)

10. The method of claim 8, wherein the PD-1 or PD-L1 immunotherapy is selected from pembrolizumab, nivolumab, cemiplumab, atezolizumab, avelumab, durvalumab, ipilimumab, tremelimumab or tisotumab; or the platinum chemotherapy is selected from carboplatin, oxaliplatin, cisplatin, nedaplatin, triplatin tetranitrate, phenanthriplatin, picoplatin, or satraplatin.

11-20. (canceled)

21. The method of claim 1, wherein the HNSCC is from a primary site in the oral cavity, pharynx, or larynx; or wherein the HNSCC is hypopharyngeal cancer, laryngeal cancer, lip or oral cavity cancer, nasopharyngeal cancer, oropharyngeal cancer, paranasal sinus or nasal cavity cancer, or salivary gland cancer.

22. The method of claim 1, wherein the effective amount of ficlatuzumab is 10 mg/kg to 100 mg/kg, or 10 mg/kg to 50 mg/kg; or wherein the effective amount of cetuximab is 250 to 700 mg/m²; or 300 to 500 mg/m².

23. (canceled)

24. The method of claim 1, wherein the effective amount of ficlatuzumab is 20 mg/kg, 15 mg/kg, or 10 mg/kg; or wherein the effective amount of cetuximab is 500 mg/m²; 400 mg/m²; or 300 mg/m².

25-27. (canceled)

28. The method of claim 1, wherein the effective amount of ficlatuzumab is administered every two weeks +/−3 days by intravenous administration; or wherein the effective amount of cetuximab is administered every two weeks +/−3 days by intravenous administration.

29-35. (canceled)

36. The method of claim 1, wherein the cetuximab and the ficlatuzumab are administered on the same day, either simultaneously or sequentially, by intravenous administration.

37-38. (canceled)

39. A method of identifying a subject with head and neck squamous cell carcinoma (HNSCC) that is suitable for a combination therapy with ficlatuzumab and cetuximab, the method comprising assessing whether the HNSCC is human papillomavirus (HPV)-negative or HPV-positive.

40. (canceled)

41. The method of claim 39, wherein the subject is identified as suitable for the combination therapy if the HNSCC is HPV-negative; the HNSCC is platinum-resistant; the subject is ineligible for platinum chemotherapy; the HNSCC is not responsive to immunotherapy with a PD-1 or PD L1 inhibitor; or the HNSCC is recurrent or metastatic;
optionally wherein:
HPV status of the HNSCC is determined by p16 immunohistochemistry or by tumoral DNA analysis, wherein the HNSCC is HPV negative when the HNSCC is classified as p16 negative or the tumoral DNA does not comprise HPV DNA;
the PD-1 or PD-L1 immunotherapy is selected from pembrolizumab, nivolumab, cemiplumab, atezolizumab, avelumab, durvalumab, ipilimumab, tremelimumab or tisotumab; or
the platinum chemotherapy is carboplatin, oxaliplatin, cisplatin, nedaplatin, triplatin tetranitrate, phenanthriplatin, picoplatin, or satraplatin.

42-64. (canceled)

65. A method of treating cancer in a subject in need thereof, the method comprising administering to the subject a therapeutically effective amount of an anti-HGF antibody or antigen binding fragment thereof and an anti-EGFR antibody or antigen binding fragment thereof; optionally wherein the cancer is a head and neck squamous cell carcinoma (HNSCC), a recurrent HNSCC, a metastatic HNSCC, or a human papillomavirus (HPV)-negative HNSCC.

66-68. (canceled)

69. The method of claim 65, wherein:
(a) the anti-HGF antibody or antigen binding fragment thereof comprises:
(i) an immunoglobulin heavy chain variable region comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3; and
(ii) an immunoglobulin light chain variable region comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6;
(ii) an immunoglobulin heavy chain variable region comprising an amino acid sequence having at least 80% identity to the amino acid sequence of SEQ ID NO: 7; and an immunoglobulin light chain variable region comprising an amino acid sequence having at least 80% identity to the amino acid sequence of SEQ ID NO: 8; or
(iii) an immunoglobulin heavy chain comprising an amino acid sequence having at least 80% identity to the amino acid sequence of SEQ ID NO: 17; and an immunoglobulin light chain comprising an amino acid sequence having at least 80% identity to the amino acid sequence of SEQ ID NO: 18;
and
(b) and wherein the anti-EGFR antibody or antigen binding fragment thereof comprises:
(i) an immunoglobulin heavy chain variable region comprising a CDRH1 comprising the amino acid sequence of SEQ ID NO: 9, a CDRH2 comprising the amino acid sequence of SEQ ID NO: 10, and a CDRH3 comprising the amino acid sequence of SEQ ID NO: 11; and an immunoglobulin light chain variable region comprising a CDRL1 comprising the amino acid sequence of SEQ ID NO: 12, a CDRL2 comprising the amino acid sequence of SEQ ID NO: 13, and a CDRL3 comprising the amino acid sequence of SEQ ID NO: 14;
(ii) an immunoglobulin heavy chain variable region comprising an amino acid sequence having at least 80% identity to the amino acid sequence of SEQ ID NO: 15; and an immunoglobulin light chain variable region comprising an amino acid sequence having at least 80% identity to the amino acid sequence of SEQ ID NO: 16; or
(iii) an immunoglobulin heavy chain comprising an amino acid sequence having at least 80% identity to the amino acid sequence of SEQ ID NO: 19; and an immunoglobulin light chain comprising an amino acid sequence having at least 80% identity to the amino acid sequence of SEQ ID NO: 20.

70. (canceled)

71. The method of claim 69, wherein:
(a) the anti-HGF antibody or antigen binding fragment thereof comprises:
(i) an immunoglobulin heavy chain variable region comprising the amino acid sequence of SEQ ID NO: 7; and an immunoglobulin light chain variable region comprising the amino acid sequence of SEQ ID NO: 8; or
(ii) an immunoglobulin heavy chain comprising the amino acid sequence of SEQ ID NO: 17; and an immunoglobulin light chain comprising the amino acid sequence of SEQ ID NO: 18;
and
(b) wherein the anti-EGFR antibody or antigen binding fragment thereof comprises:
(i) an immunoglobulin heavy chain variable region comprising the amino acid sequence of SEQ ID NO: 15; and an immunoglobulin light chain variable region comprising the amino acid sequence of SEQ ID NO: 16; or
(ii) an immunoglobulin heavy chain comprising the amino acid sequence of SEQ ID NO: 19; and an immunoglobulin light chain comprising the amino acid sequence of SEQ ID NO: 20.

72-73. (canceled)

74. The method of claim 65, wherein:
(a) the anti-HGF antibody or antigen binding fragment thereof is, or is derived from, a chimeric antibody, a human antibody, or a humanized antibody; or
(b) the anti-EGFR antibody or antigen binding fragment thereof is, or is derived from, a chimeric antibody, a human antibody, or a humanized antibody.

75. The method of claim 65, wherein:
(a) the anti-HGF antibody or antigen binding fragment thereof is selected from the group consisting of: a Fab, a Fv, a scFv, a Fab′, and a (Fab′)2, or
(b) the anti-EGFR antibody or antigen binding fragment thereof is selected from the group consisting of: a Fab, a Fv, a scFv, a Fab′, and a (Fab′)2.

76. The method of claim 65, wherein:
(a) the anti-HGF antibody is selected from the group consisting of: rilotumumab, ficlatuzumab, and combinations thereof; or
(b) the anti-EGFR antibody is selected from the group consisting of: cetuximab, futuximab, imgatuzumab, matuzumab, necitumumab, nimotuzumab, panitumumab, amivantamab, zalutumumab, and combinations thereof.

77. The method of claim 76, wherein:
(a) the anti-HGF antibody is ficlatuzumab; or
(b) the anti-EGFR antibody is cetuximab.

78-88. (canceled)

89. The method of claim 65, wherein the anti-HGF antibody or antigen binding fragment thereof and the anti-EGFR antibody or antigen binding fragment thereof are each administered about every week, about every two weeks, about every three weeks, or about every four weeks.

90. (canceled)

91. The method of claim 65, wherein the anti-HGF antibody or antigen binding fragment thereof is administered at about 2 mg/kg, about 5 mg/kg, about 10 mg/kg, about 15 mg/kg, about 20 mg/kg, or about 25 mg/kg; and wherein the anti-EGFR antibody or antigen binding fragment thereof is administered at about 200 mg/m², about 250 mg/m², about 300 mg/m², about 400 mg/m², about 500 mg/m², about 600 mg/m², about 700 mg/m², or about 800 mg/m².

92. The method of claim 91, wherein the anti-HGF antibody or antigen binding fragment thereof is administered at about 20 mg/kg; and wherein the anti-EGFR antibody or antigen binding fragment thereof is administered at about 500 mg/m².

93-95. (canceled)

96. The method of claim 65, wherein the anti-HGF antibody is ficlatuzumab; and the anti-EGFR antibody is cetuximab; and wherein the method comprises administering to the subject (i) 20 mg/kg ficlatuzumab, and (ii) 500 mg/m² cetuximab every two weeks.

97. A composition comprising the anti-HGF antibody or antigen binding fragment thereof and the anti-EGFR antibody or antigen binding fragment thereof according to claim 69; wherein the composition further comprises a pharmaceutically acceptable carrier.

98. (canceled)


SEQUENCE LISTING (46 sequences)

SEQ ID NO: 1 - protein, 5 aa, synthetic construct
TYWMH

SEQ ID NO: 2 - protein, 17 aa, synthetic construct
EINPTNGHTNYNQKFQG

SEQ ID NO: 3 - protein, 9 aa, synthetic construct
NYVGSIFDY

SEQ ID NO: 4 - protein, 11 aa, synthetic construct
KASENVVSYVS

SEQ ID NO: 5 - protein, 7 aa, synthetic construct
GASNRES

SEQ ID NO: 6 - protein, 9 aa, synthetic construct
GQSYNYPYT

SEQ ID NO: 7 - protein, 118 aa, synthetic construct
QVQLVQPGAEVKKPGTSVKLSCKASGYTFTTYWMHWVRQAPGQGLEWIGEINPTNGHTNYNQKFQGRATLTVDKSTSTAYMELSSLRSEDTAVYYCARNYVGSIFDYWGQGTLLTVSS

SEQ ID NO: 8 - protein, 107 aa, synthetic construct
DIVMTQSPDSLAMSLGERVTLNCKASENVVSYVSWYQQKPGQSPKLLIYGASNRESGVPDRFSGSGSATDFTLTISSVQAEDVADYHCGQSYNYPYTFGQGTKLEIK

SEQ ID NO: 9 - protein, 5 aa, synthetic construct
NYGVH

SEQ ID NO: 10 - protein, 16 aa, synthetic construct
VIWSGGNTDYNTPFTS

SEQ ID NO: 11 - protein, 11 aa, synthetic construct
ALTYYDYEFAY

SEQ ID NO: 12 - protein, 11 aa, synthetic construct
RASQSIGTNIH

SEQ ID NO: 13 - protein, 7 aa, synthetic construct
YASESIS

SEQ ID NO: 14 - protein, 9 aa, synthetic construct
QQNNNWPTT

SEQ ID NO: 15 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 16 - protein, 108 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 17 - protein, 448 aa, synthetic construct
QVQLVQPGAEVKKPGTSVKLSCKASGYTFTTYWMHWVRQAPGQGLEWIGEINPTNGHTNYNQKFQGRATLTVDKSTSTAYMELSSLRSEDTAVYYCARNYVGSIFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
DIVMTQSPDSLAMSLGERVTLNCKASENVVSYVSWYQQKPGQSPKLLIYGASNRESGVPDRFSGSGSATDFTLTISSVQAEDVADYHCGQSYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
IYYWS

SEQ ID NO: 22 - protein, 16 aa, synthetic construct
YVYYSGSTNYNPSLKS

SEQ ID NO: 23 - protein, 12 aa, synthetic construct
GGYDFWSGYFDY

SEQ ID NO: 24 - protein, 11 aa, synthetic construct
RASQSVDSNLA

SEQ ID NO: 25 - protein, 7 aa, synthetic construct
GASTRAT

SEQ ID NO: 26 - protein, 10 aa, synthetic construct
QQYINWPPIT

SEQ ID NO: 27 - protein, 139 aa, synthetic construct
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSS

SEQ ID NO: 28 - protein, 128 aa, synthetic construct
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIK

SEQ ID NO: 29 - protein, 446 aa, synthetic construct
QVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 215 aa, synthetic construct
EIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 449 aa, synthetic construct
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 213 aa, synthetic construct
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 36 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 37 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct; identical to SEQ ID NO: 37

SEQ ID NO: 41 - protein, 221 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 42 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 140 aa, synthetic construct
LSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVP

SEQ ID NO: 44 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITREVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 223 aa, synthetic construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 46 - protein, 212 aa, synthetic construct
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE